SEQUENCE LISTING (15 sequences)

SEQ ID NO: 1 - protein, 10 aa, Artificial Sequence
  source: /note=“Description of Artificial Sequence Synthetic peptide”
GYTFTSSYIN

SEQ ID NO: 2 - protein, 17 aa, Artificial Sequence
  source: /note=“Description of Artificial Sequence Synthetic peptide”
TINPVSGSTSYAQKFQG

SEQ ID NO: 3 - protein, 6 aa, Artificial Sequence
  source: /note=“Description of Artificial Sequence Synthetic peptide”
GGWFDY

SEQ ID NO: 4 - protein, 14 aa, Artificial Sequence
  source: /note=“Description of Artificial Sequence Synthetic peptide”
TGTSSDVGSYNYVN

SEQ ID NO: 5 - protein, 11 aa, Artificial Sequence
  source: /note=“Description of Artificial Sequence Synthetic peptide”
LMIYGVSKRPS

SEQ ID NO: 6 - protein, 10 aa, Artificial Sequence
  source: /note=“Description of Artificial Sequence Synthetic peptide”
GTFAGGSYYG

SEQ ID NO: 7 - protein, 217 aa, Artificial Sequence
  source: /note=“Description of Artificial Sequence Synthetic polypeptide”
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 445 aa, Artificial Sequence
  source: /note=“Description of Artificial Sequence Synthetic polypeptide”
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 111 aa, Artificial Sequence [sequence omitted]

SEQ ID NO: 10 - protein, 115 aa, Artificial Sequence [sequence omitted]

SEQ ID NO: 11 - protein, 6 aa, Homo sapiens
WLDDFN

SEQ ID NO: 12 - protein, 9 aa, Homo sapiens
GCWLDDFNC

SEQ ID NO: 13 - protein, 11 aa, Homo sapiens
KGCWLDDFNCY

SEQ ID NO: 14 - protein, 5 aa, Homo sapiens
EQDKR

SEQ ID NO: 15 - protein, 15 aa, Homo sapiens
CEGEQDKRLHCYASW

(19) United States
(12) Patent Application Publication
(10) Pub. No.: US 2022/0340668 A1
(43) Pub. Date: Oct. 27, 2022

(54) TREATMENT OF LIVER DISEASE OR DISORDER COMPRISING ACTRII RECEPTOR ANTAGONISTS

(71) Applicant: Novartis AG, Basel (CH)

(72) Inventors: Laura COLEMAN, Boston, MA (US); C. Daniel MEYERS, Littleton, MA (US); Ronenn ROUBENOFF, Basel (CH); Estelle TRIFILIEFF, Basel (CH)

(21) Appl. No.: 17/639,862

(22) PCT Filed: Sep. 1, 2020
(86) PCT No.: PCT/IB2020/058114

§ 371 Date: Mar. 2, 2022

Related U.S. Application Data
(60) Provisional application No. 62/895,107, filed on Sep. 3, 2019.

(51) Int. Cl. C07K 16/28; A61P 1/16; A61P 3/10

(52) U.S. Cl. CPC C07K 16/2863; A61P 1/16; A61P 3/10; C07K 2317/24; C07K 2317/76

(57) ABSTRACT

The present disclosure provides an ActRII antagonist, e.g., the ActRIIA and/or ActRIIB antagonist, e.g., an anti-ActRII receptor antibody or antigen-binding fragment thereof, e.g., bimagrumab, for treating or preventing liver disease or disorder in a subject in need thereof. The present disclosure also relates to pharmaceutical combinations comprising such ActRII antagonists and at least one further therapeutic agent in the treatment or prevention of liver disease or disorder.

### SEQUENCE LISTING

The instant application contains a Sequence Listing which has been submitted electronically in ASCII format and is hereby incorporated by reference in its entirety. Said ASCII copy, created on Jul. 14, 2020, is named PAT058683-WO-PCT_SL.txt and is 10,864 bytes in size.

### TECHNICAL FIELD

The present disclosure relates to activin receptor type II (ActRII) antagonists, e.g., molecules capable of antagonizing the binding of activins, growth differentiation factors (GDFs), bone morphogenetic proteins (BMPs) and/or myostatin to the human ActRII receptor, e.g., an antagonist antibody to ActRIIA and/or ActRIIB, e.g., an anti-ActRII receptor antibody, e.g., bimagrumab for use in methods of preventing or treating liver disease or disorder. The present disclosure also relates to methods of preventing or treating liver disease or disorder by administering to a subject in need thereof a therapeutically effective amount of an ActRII receptor antagonist.

The present disclosure also relates to a pharmaceutical combination comprising a) an activin receptor type II (ActRII) antagonist, e.g., molecules capable of antagonizing the binding of activins, growth differentiation factors (GDFs), bone morphogenetic proteins (BMPs) and/or myostatin to the human ActRII receptor, e.g., an ActRIIA and/or ActRIIB antagonist, e.g., an anti-ActRII receptor antibody e.g., bimagrumab and b) at least one further therapeutic agent, optionally in the presence of a pharmaceutically acceptable carrier and pharmaceutical compositions comprising them. Furthermore, the disclosure is directed to the use of such pharmaceutical combinations for treating or preventing liver diseases or disorders, as well as compositions, methods, uses and regimens involving such combinations.

### BACKGROUND OF THE DISCLOSURE

Nonalcoholic fatty liver disease (NAFLD) is one of the the most common cause of chronic liver disease in the Western world (Ratziu et al J Hepatol. 2010 August; 53(2):372-84.). The main stages of NAFLD are 1—simple fatty liver (hepatic steatosis), where excessive fat accumulates in liver cells via the process of steatosis (i.e., abnormal retention of lipids within a cell); 2—non-alcoholic steatohepatitis (NASH), a more serious form of NAFLD, where hepatic steatosis converts into a progressive inflammation of the liver (hepatitis), called steatohepatitis; 3—fibrosis, where there is a persistent inflammation in the liver resulting in the generation of fibrous scar tissue around the liver cells and blood vessels; and 4-cirrhosis, where this damage is permanent and can lead to liver failure and liver cancer (hepatocellular carcinoma; HCC). Liver transplantation is the only treatment for advanced cirrhosis with liver failure, and transplantation is increasingly performed in persons suffering from NASH.

Steatosis, lobular inflammation, and hepatocellular ballooning are all necessary components for the diagnosis of NASH and fibrosis is also typically observed. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. NASH is highly associated with metabolic syndrome and Type 2 diabetes mellitus.

Furthermore, cardiovascular mortality is an important cause of death in NASH patients.

The Activin type 2 receptors (ActRIIA and ActRIIB, collectively abbreviated as ActRII) modulate signals for ligands belonging to the transforming growth factor beta (TGF-β) superfamily such as myostatin, GDF-11, and activins. Myostatin, activin A, and GDF-11 are negative regulators of skeletal muscle growth, acting via the ActRII receptor signaling pathway to inhibit muscle protein synthesis and myocyte differentiation and proliferation.

Bimagrumab (international nonproprietary name (INN) 9711, also known as BYM338 or MOR08159), a recombinant human, monoclonal antibody binds competitively to ActRII with greater affinity than its natural ligands myostatin or activin. Bimagrumab is disclosed in WO2010/125003, which is incorporated by reference herein. The Bimagrumab sequences disclosed in WO2010/125003 are listed in table 1. Bimagrumab has shown a significant increase in skeletal muscle mass in healthy volunteers, in patients with sporadic inclusion body myositis (sIBM), and in patients with sarcopenia. In previous studies, a single dose of bimagrumab caused an increase in thigh muscle volume measured by magnetic resonance imaging of approximately 6% after 10 weeks in healthy lean adults compared to placebo, and reduced fat mass to a comparable extent (Roubenoff and Papanicolaou, New treatments for muscle wasting: an update on bimagrumab and other treatments. Abstract at ICFSR 2015). A single dose of bimagrumab resulted in a profound impact on body composition with a maximal reduction in fat mass of ˜8% and an increase in lean mass of ˜3% (DXA), in overweight/obese pre-diabetic patients (Canto et al, Diabetes Obes Metab. 2018 January; 20(1):94-102).

Currently, there are no approved drugs for the prevention or treatment of NAFLD, including NASH. Treatment of liver related diseases or disorders, in particular liver diseases such as NAFLD or NASH, hence represent a substantial, unmet medical need.

### SUMMARY OF THE DISCLOSURE

The present disclosure relates, in part, to the finding that direct inhibition of myostatin or activin binding to their receptors ActRII (preferably ActRIIB and ActRIIA, or ActRIIA or ActRIIB either alone) by administration of ActRII binding antibodies significantly reduces hepatic fat.

Accordingly, the present disclosure is directed to an activin receptor type II (ActRII) antagonists, e.g., molecules capable of antagonizing the binding of activins, growth differentiation factors (GDFs), bone morphogenetic proteins (BMPs) and/or myostatin to the human ActRII receptor, preferably an ActRIIA and/or ActRIIB antagonist, preferably an antagonist antibody to ActRIIA and/or ActRIIB, most preferably bimagrumab, for use in preventing or treating liver disease or disorder. The present disclosure is also directed to methods of preventing or treating liver disease or disorder by administering to a subject in need thereof a therapeutically effective amount of an activin receptor type II (ActRII) antagonists, e.g., molecules capable of antagonizing the binding of activins, growth differentiation factors (GDFs), bone morphogenetic proteins (BMPs) and/or myostatin to the human ActRII receptor, preferably an antagonist antibody to ActRIIA and/or ActRIIB, most preferably bimagrumab.

As a potent inhibitor of ActRII, bimagrumab blocks the effects of myostatin, activin A, GDF11, and possibly other ligands working through those receptors (Lach-Trifilieff et al, Mol Cell Biol. 2014 February; 34(4):606-18).

The present disclosure therefore provides an activin receptor type II antagonist, preferably a ActRIIA and/or ActRIIB antagonist, and more preferably an anti-ActRII receptor antibody, most preferably bimagrumab, for use in reducing hepatic fat.

The present disclosure therefore provides an activin receptor type II antagonist, preferably an ActRIIA and/or ActRIIB antagonist, and more preferably an anti-ActRII receptor antibody, most preferably bimagrumab, for use in the treatment or prevention of liver disease or disorder.

In a similar aspect the present disclosure provides an activin receptor type II antagonist, preferably an ActRIIA and/or ActRIIB antagonist, and even more preferably an anti-ActRII receptor antibody, most preferably bimagrumab, for use in the treatment or prevention of liver disease or disorder in a patient, whereby liver fat is reduced.

In a similar aspect the present disclosure provides an activin receptor type II, preferably an ActRIIA and/or ActRIIB antagonist, preferably an anti-ActRII receptor antibody, most preferably bimagrumab, for use in the treatment, prevention or reduction of comorbidities related to liver disease or disorder, particularly those related to increased liver fat mass.

The present disclosure further provides specific dose regimen for the myostatin receptor antagonist bimagrumab for use herein.

The disclosure also relates to pharmaceutical combinations comprising a) an activin receptor type II (ActRII) antagonist, e.g., molecules capable of antagonizing the binding of activins, growth differentiation factors (GDFs), bone morphogenetic proteins (BMPs) and/or myostatin to the human ActRII receptor, preferably an ActRIIA and/or ActRIIB antagonist, and more preferably an anti-ActRII receptor antibody, most preferably bimagrumab, and b) at least one further therapeutic agent, optionally in the presence of a pharmaceutically acceptable carrier, for use in the treatment or prevention of liver disease or disorder and pharmaceutical compositions comprising them.

Further features and advantages of the disclosure will become apparent from the following detailed description of the disclosure.

### BRIEF DESCRIPTION OF THE DRAWINGS

FIG. 1A-D. Body weight (FIG. 1A), lean mass (FIG. 1B), total fat mass (FIG. 1C) and % liver fat (FIG. 1D) in mice on normal diet (ND) (Group #3), and with diet induced NASH (HF/NASH) with (+CDD866) (Group #1) and without (+control (Ctrl) (Group #2) treatment with CDD866 over 20 weeks.

FIG. 2A-C. PicroSirius Red (PSR) staining of liver sections at week 20 in mice on normal diet (ND) and with diet induced NASH (HF/NASH) with (+CDD866) and without (+control (Ctrl) treatment with CDD866 (murinized BYM338). Panel A) PSR stained area in mm2, panel B) representative image of PSR stained liver sections, panel C) histopathology score.

FIG. 2D-E. Immunohistological analysis for inflammation (IBA1) and fibrosis (anti-smooth muscle antibodies (aSMA) in mice on normal diet (ND), and with diet induced NASH with and without treatment with CDD866. Panel D) aSMA stained total tissue area and representative image of aSMA stained liver sections, panel E) total number of IBA1-positive hepatic crown like structures.

FIG. 2F. Micro- and macrovesicular steatosis in hematoxylin and eosin (H&E) stained liver section. Histopathology score and representative H&E image.

FIG. 3A-D. Gene expression and serum analysis in mice on normal diet (ND), and with diet induced NASH with and without treatment with CDD866. Panel A) Levels of gene expression in markers of fibrosis, Panel B) Levels of gene expression in markers of inflammation, Panel C) serum levels of TIMP1, Panel D) serum levels of PIIINP.

FIG. 4. Levels of aspartate aminotransferase (AST) and gamma-glutamyl transferase (GGT) in mice on normal diet (ND), and with diet induced NASH with and without treatment with CDD866.

FIG. 5A-E. Body weight (FIG. 5A), lean mass (FIG. 5B), total fat mass (FIG. 5C), weight of white adipose tissue (WAT) (FIG. 5D) and weight of brown adipose tissue (BAT) (FIG. 5E) in control mice (Group #3) and in mice with CCl4 induced liver fibrosis with (+CDD866) (Group #1) and without (+vehicle) (Group #2) treatment over 28 days.

FIG. 6A-B. PicroSirius Red (PSR) staining of liver sections at week 4 in control mice and in mice with CCl4 induced liver fibrosis with (+CDD866) and without (+vehicle) treatment. Panel A) PSR stained area in mm2, panel B) representative image of PSR stained liver sections

FIG. 6C-D. Immunohistological analysis of fibrosis (anti-smooth muscle antibodies (aSMA) at week 4 in control mice and in mice with CCl4 induced liver fibrosis with (+CDD866) and without (+vehicle) treatment. Panel C) aSMA stained total tissue area, panel D) representative image of aSMA stained liver sections.

FIG. 6E. Serum analysis of TIMP-1 and PIIINP in control mice and in mice with CCl4 induced liver fibrosis with (+CDD866) and without (+vehicle) treatment at week 4.

FIG. 7. Levels of gene expression in markers of fibrosis in control mice and in mice with CCl4 induced liver fibrosis with (+CDD866) and without (+vehicle) treatment at week 4.

FIG. 8. Total body fat mass assessed by DXA in subjects receiving 10 mg/kg BYM338 or placebo from baseline to end of study (EOS) at week 56.

FIG. 9A-B. Anthropometrics in subjects receiving 10 mg/kg BYM338 or placebo from baseline to end of study (EOS) at week 56. Panel A) Body weight in kg, panel B) BMI (kg/m2).

FIG. 10. Hepatic fat fraction in % in subjects receiving 10 mg/kg BYM338 or placebo at baseline, week 24 and week 48.

### DETAILED DESCRIPTION OF THE DISCLOSURE

The present disclosure relates to methods of treating or preventing a liver disease or disorder by administering to a subject in need thereof an effective amount of an activin receptor type II (ActRII) antagonists, e.g., molecules capable of antagonizing the binding of activins, growth differentiation factors (GDFs), bone morphogenetic proteins (BMPs) and/or myostatin to the human ActRII receptor, preferably an antagonist antibody to ActRIIA and/or ActRIIB, most preferably bimagrumab. Accordingly, in one aspect provided is a method of preventing or treating liver disease or disorder comprising administering to a subject in need thereof an effective amount of bimagrumab. Also provided is an activin receptor type II (ActRII) antagonists, e.g., molecules capable of antagonizing the binding of activins, growth differentiation factors (GDFs), bone morphogenetic proteins (BMPs) and/or myostatin to the human ActRII receptor, preferably an antagonist antibody to ActRIIA and/or ActRIIB, most preferably bimagrumab for use in treating or preventing liver disease or disorder. In one embodiment of any method or use of the disclosure, said activin receptor type II (ActRII) antagonists thereof is an ActRIIA-binding and/or ActRIIB-binding antibody. In some embodiments of any and/or all of the methods or uses described herein, the ActRIIA-binding and/or ActRIIB-binding antibody is a neutralizing antibody.

### Definitions

In order that the present disclosure may be more readily understood, certain terms are first defined. Additional definitions are set forth throughout the detailed description.

All patents, published patent applications, publications, references and other material referred to herein are incorporated by reference herein in their entirety.

As used herein, the term “comprising” encompasses “including” as well as “consisting of” e.g. a composition “comprising” X may consist exclusively of X or may include something additional, e.g., X+Y.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical value disclosed throughout can be “about” that numerical value even without specifically mentioning the term “about.”

As used herein, the term “baseline” refers to a subject's state or the degree of a condition, e.g. a disease, or one or more parameters associated with the state of a patient, observed before treatment, e.g., before administration of a compound, e.g., before administration of ActRII antagonist, optionally in combination with at least one further therapeutic agent, according to the present disclosure.

As used herein, the term “administering” in relation to a compound, e.g., the ActRII antagonist, optionally in combination with at least one further therapeutic agent, is used to refer to delivery of that compound by any route of delivery.

As used herein, the word “substantially” does not exclude “completely,” e.g., a composition which is “substantially free” from Y may be completely free from Y. Where necessary, the word “substantially” may be omitted from the definition of the disclosure.

As used herein, the term “pharmaceutically acceptable” means a nontoxic material that does not substantially interfere with the effectiveness of the biological activity of the active ingredient(s).

The terms “ActRIIA” and “ActRIIB” refer to Activin receptors. Activins signal through a heterodimeric complex of receptor serine kinases which include at least two type I (I and IB) and two type II (IIA and IIB, aka ACVR2A and ACVR2B) receptors. These receptors are all transmembrane proteins, composed of a ligand-binding extracellular domain with a cysteine-rich region, a transmembrane domain, and a cytoplasmic domain with predicted serine/threonine specificity. Type I receptors are essential for signaling while type II receptors are required for binding ligands and for expression/recruitment of type I receptors. Type I and II receptors form a stable complex after ligand binding resulting in the phosphorylation of type I receptors by type II receptors. The activin receptor II B (ActRIIB) is a receptor for myostatin. The activin receptor II A (Act RIIA) is also a receptor for myostatin. The term ActRIIB or Act IIB receptor refers to human ActRIIB (AAC64515.1, GI:3769443). Research grade polyclonal and monoclonal anti-ActRIIB antibodies are known in the art, such as those made by R&D Systems®, MN, USA. Of course, antibodies could be raised against ActRIIB from other species and used to treat pathological conditions in those species.

By “ActRII binding molecule” is meant any molecule capable of binding to the human ActRII receptors ActRIIA and/or ActRIIB either alone or associated with other molecules. The binding reaction may be shown by standard methods (qualitative assays) including, for example, a binding assay, competition assay or a bioassay for determining the inhibition of ActRII receptor binding to myostatin or any kind of binding assays, with reference to a negative control test in which an antibody of unrelated specificity, but ideally of the same isotype, e.g., an anti-CD25 antibody, is used. Non-limiting examples of ActRII receptor binding molecules include small molecules such as aptamers or other nucleic acid molecules designed and/or subject to bind the receptor, ligand decoys, and antibodies to the ActRII receptor as produced by B cells or hybridomas and chimeric, CDR-grafted or human antibodies or any fragment thereof, e.g., F(ab′)2 and Fab fragments, as well as single chain or single domain antibodies. Preferably the ActRII receptor binding molecule antagonizes (e.g., reduces, inhibits, decreases, delays) the binding of natural ligands to the ActRII receptor. In some embodiments of the disclosed methods, regimens, kits, processes and uses, an ActRIIB receptor binding molecule is employed.

A “signaling activity” refers to a biochemical causal relationship generally initiated by a protein-protein interaction such as binding of a growth factor to a receptor, resulting in transmission of a signal from one portion of a cell to another portion of a cell. In general, the transmission involves specific phosphorylation of one or more tyrosine, serine, or threonine residues on one or more proteins in the series of reactions causing signal transduction. Penultimate processes typically include nuclear events, resulting in a change in gene expression.

As used herein, the term “patient” is used interchangeably with the term “subject” and refers to a human patient.

As used herein, a subject is “in need of” a treatment if such subject who is diseased with the condition (i.e. disease or disorder) of interest and who would benefit biologically, medically or in quality of life from such treatment.

The term “treating or preventing” includes the administration of a compound, e.g., the ActRII antagonist, suitably bimagrumab, optionally in combination with at least one further therapeutic agent, to prevent or delay the onset of the symptoms, complications, or biochemical indicia of a disease (e.g., liver disease or disorder), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, the term “prevent”, “preventing” or “prevention” in connection to a disease or disorder refers to the prophylactic treatment of a subject who is at risk of developing a condition (e.g., a specific disease or disorder or clinical symptom thereof such as liver disease or disorder) resulting in a decrease in the probability that the subject will develop the condition.

The terms “treat”, “treating” and “treatment” refer to both therapeutic treatment and prophylactic or preventive measures, wherein the object is to ameliorate the disease or disorder (i.e., slowing or arresting or reducing the development of the disease or at least one of the clinical symptoms thereof) by alleviating or ameliorating at least one physical parameter including those which may not be discernible by the patient. The terms “treat”, “treating” or “treatment” also refer to modulating the disease or disorder, either physically, (e.g., stabilization of a discernible symptom), physiologically, (e.g., stabilization of a physical parameter), or both and/or to preventing or delaying the onset or development or progression of the disease or disorder.

For example, “treating NASH” or “treating NAFLD” may refer to ameliorating, alleviating or modulating at least one of the symptoms or pathological features associated with NASH/NAFLD; e.g., hepatosteatosis, hepatocellular ballooning, hepatic inflammation and fibrosis; e.g. may refer to slowing progression, reducing or stopping at least one of the symptoms or pathological features associated with NASH/NAFLD, e.g., hepatosteatosis, hepatocellular ballooning, hepatic inflammation and fibrosis. It may also refer to preventing or delaying liver cirrhosis or a need for liver transplantation, e.g., slow the progress of, halt, or reverse disease progression and improve clinical outcomes (i.e., prevent progression to cirrhosis and cirrhosis complications, reduce the need for liver transplantation, and improve survival)

As used herein, the term NAFLD may encompass the different stages of the disease: hepatosteatosis or non-alcoholic fatty liver (NAFL), NASH, NASH with fibrosis and NASH with cirrhosis. NASH is usually characterized by hepatic steatosis, hepatocellular ballooning and lobular inflammation. NAFL is characterized by the accumulation of triacylglycerol (TAG) in the liver. Hepatic steatosis is defined as intrahepatic TAG of at least 5% of liver weight or 5% of hepatocytes containing lipid vacuoles in the absence of a secondary contributing factor such as excess alcohol intake, viral infection, or drug treatments.

Also “treating” NASH may refer to slow the progress of, halt, or reverse disease progression and improve clinical outcomes, i.e., prevent progression to cirrhosis and resolution of steatohepatitis and no worsening of liver fibrosis on NASH clinical research network (CRN) histological score.

The treatment of NASH includes:
“Resolution of steatohepatitis” is defined as absence of fatty liver disease or isolated or simple steatosis without steatohepatitis and a NAS score of 0-1 for inflammation, 0 for ballooning, and any value for steatosis; cirrhosis complications, reduction in the need for liver transplantation, and improved survival;
Or improvement in liver fibrosis greater than or equal to one stage (NASH CRN histological score) and no worsening of steatohepatitis (e.g. defined as no increase in NAS for ballooning, inflammation, or steatosis);
Or both resolution of steatohepatitis and improvement in fibrosis (as defined above).

“Treating” or “treatment” of NAFLD or NASH in a human includes one or more of:
a) Reducing the risk of developing NAFLD or NASH, i.e., causing clinical symptoms of NAFLD or NASH not to develop in a subject who may be predisposed to NAFLD or NASH
b) Inhibiting NAFLD or NASH, i.e., arresting or reducing the development of NALFD or NASH or its clinical symptoms; and
c) Relieving NAFLD or NASH, i.e., causing regression, reversal, or amelioration of the NAFLD or NASH or reducing number, frequency, duration or severity of its clinical symptoms.

As used herein, “NAS score” refers to the NAFLD activity score, a widely used histological grading and staging system for NAFLD (Kleiner et al, Hepatology. 2005 June; 41(6):1313-21).

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week, and also include less frequent administration, e.g., as described herein. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, the term “a therapeutically effective amount” of the compound of the present disclosure refers to an amount of the compound of the present disclosure that will elicit the biological or medical response of a subject, for example, ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to at least partially alleviating, inhibiting, preventing and/or ameliorating a condition associated with liver disease or disorder.

As herein defined, “combination” refers to either a fixed combination in one unit dosage form (e.g., capsule, tablet, sachet or vial), free (i.e. non-fixed) combination, or a kit of parts for the combined administration where an ActRII antagonist, such as bimagrumab, and the one or more additional therapeutic agents may be administered independently at the same time or separately within time intervals, especially where these time intervals allow that the combination partners show a cooperative, e.g. synergistic effect.

The terms “co-administration” or “combined administration” or the like as utilized herein are meant to encompass administration of an additional therapeutic agent to a single subject in need thereof (e.g. a subject), and the additional therapeutic agent are intended to include treatment regimens in which the ActRII antagonist, such as bimagrumab, and additional therapeutic agent are not necessarily administered by the same route of administration and/or at the same time. Each of the components of the combination of the present disclosure may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients are administered to a subject simultaneously in the form of a single entity or dosage.

The term “free combination (non-fixed combination)” means that the active ingredients as defined herein are administered to a subject as separate entities either simultaneously, concurrently or sequentially with no specific time limits, and in any order, wherein such administration provides therapeutically effective levels of the compounds in the subject's body. In particular, reference to the combination comprising a) an activin receptor type II (ActRII) antagonists, e.g., molecules capable of antagonizing the binding of activins, growth differentiation factors (GDFs), bone morphogenetic proteins (BMPs) and/or myostatin to the human ActRII receptor, preferably an antagonist antibody to ActRIIA and/or ActRIIB, most preferably bimagrumab, and b) at least one additional therapeutic agent as used herein (e.g., in any of the embodiments or in any of the claims herein), refers to a “non-fixed combination” and may be administered independently at the same time or separately within time intervals.

By “simultaneous administration”, it is meant that the active ingredients as defined herein, are administered on the same day. The active ingredients can be administered at the same time (for fixed or free combinations), or one at a time (for free combinations).

According to the disclosure, “sequential administration”, may mean that during a period of two or more days of continuous co-administration only one of active ingredients as herein defined, is administered on any given day.

By “overlapping administration”, it is meant that during a period of two or more days of continuous co-administration, there is at least one day of simultaneous administration and at least one day when only one of active ingredients as herein defined, is administered.

By “continuous administration”, it is meant a period of co-administration without any void day. The continuous administration may be simultaneous, sequential, or overlapping, as described above.

The term “dose” refers to a specified amount of a drug administered at one time. The dose would, for example, be declared on a product package or in a product information leaflet.

The term “obesity” is based on BMI for both youth and adults, but the definitions are not directly comparable. Among adults, there is a set cut point based on health risk, while among children the definition is statistical and is based on a comparison to a reference population. BMI was calculated as weight in kilograms divided by height in meters squared, rounded to one decimal place. Obesity in adults is defined as a BMI of greater than or equal to 30 kg/m2.

Obesity in youth is defined as a BMI of greater than or equal to the age- and sex-specific 95th percentile of the 2000 CDC growth charts.

The term “overweight condition” is based on a BMI≥25−<30 kg/m2.

Overweight condition may also be associated with at least one additional risk factor for fatal diseases (stroke, MI, heart failure, sudden death) such as diabetes, hypertension, family history of premature coronary artery disease, and the like.

Because different subjects can have same BMI but different percentage of fat and muscle mass, BMI is not always a good index to classify overweight and obesity. A high percentage of muscle mass can lead to a high BMI even with a small percentage of fat; in this case the subject may be wrongly considered overweight or obese, based on the BMI classification. Other indexes in addition to BMI are used, namely waist circumference and a body shape index (ABSI): imaging, by Dual-energy X-ray absorptiometry (DXA or DEXA) and magnetic resonance imaging (MRI), is often used to quantify the percentage of muscle, fat and the fat distribution.

The term “body composition” is used herein to describe the percentages of fat and muscle in human bodies. Because muscular tissue takes up less space in our body than fat tissue, our body composition, as well as our weight, determines leanness.

“Lean body mass” is a component of body composition, calculated by subtracting body fat weight from total body weight:total body weight is lean plus fat.

In equations:

LBM=BW−BF

Lean body mass equals body weight minus body fat

LBM+BF=BW

Lean body mass plus body fat equals body weight

The percentage of total body mass that is lean is usually not quoted—it would typically be 60-90%. Instead, the body fat percentage, which is the complement, is computed, and is typically 10-40%. The lean body mass (LBM) has been described as an index superior to total body weight for prescribing proper levels of medications and for assessing metabolic disorders, as body fat is less relevant for metabolism.

The term “fat mass” refers to that portion of the human body that is composed strictly of fat. It can be measured with DXA, MRI or bioelectrical impedance techniques.

The term “central adiposity refers to the following: Obesity is defined as a condition of abnormal or excessive fat accumulation in adipose tissue. The amount of excess fat in absolute terms, and its regional distribution between different fat depots both play an important role in determining the health impact of obesity. Obesity can be categorized into central/android obesity and peripheral/gynoid obesity, android obesity being more typical for men while gynoid obesity being more characteristic for women.

There is substantial evidence in the literature arguing that not all obesity are associated with adverse metabolic profile and increases in cardiovascular risk. In fact, body fat distribution (i.e. relative presence of abdominal versus peripheral fat mass) was deemed as a better indicator of the metabolic and cardiovascular risks than the degree of obesity per se. In men, who tend to accumulate fat in the truncal region, increasing BMI is associated with increasing CV risk, while in women BMI is a generally poor indicator/surrogate of cardiovascular risk.

Trunk fat mass can be subdivided into subcutaneous (SC) fat (in the abdominal wall) and visceral adipose tissue (in the intra-abdominal cavity). Subcutaneous and visceral fat differ significantly in terms of their anatomy, cellular composition, endocrine function and cellular regulation. VAT compared with SC is more cellular, vascular, innervated and infiltrated by inflammatory and immune cells, which translate to a higher metabolic activity and increased release of pro-inflammatory cytokines with direct and indirect implications for insulin resistance, type 2 diabetes, and the risk of cardiovascular disease. In contrast, subcutaneous fat mass, especially fat depots of the thigh and buttocks, were associated with constitutive secretion of adiponectin conferring insulin sensitizing, anti-inflammatory and anti-atherogenic effects. Low-grade inflammation has been associated with muscle wasting, which in turn may further worsen insulin sensitivity and add to the relative risk of developing type 2 diabetes.

Therefore, an imbalance between central and peripheral fat depots (central adiposity) even without manifest obesity (i.e. BMI <30 kg/m2) can be linked to pronounced insulin resistance, metabolic alterations and systemic low-grade inflammation collectively driving accelerated atherogenesis.

The term “type II diabetes” referred as type 2 diabetes, previously referred to as “non-insulin-dependent diabetes” or “adult-onset diabetes,” accounts for 90-95% of all diabetes, encompasses individuals who have insulin resistance and usually relative (rather than absolute) insulin deficiency. At least initially, and often throughout their lifetime, these individuals may not need insulin treatment to survive. There are various causes of type 2 diabetes.

“Insulin sensitivity” describes how sensitive the body is to the effects of insulin. Someone said to be insulin sensitive will require smaller amounts of insulin to lower blood glucose levels than someone who has low sensitivity. Insulin sensitivity varies from person to person and doctors can perform tests to determine how sensitive an individual is to insulin.

“Insulin resistance” is defined as a condition of tolerance to insulin, making the hormone less effective, causing decreased glucose uptake in muscle tissue that result in impaired glucose oxidation and glycogen synthesis, and a deficient suppression of hepatic glucose production in the liver. In obese, increased visceral fat mass with elevation of plasma free fatty acid (FFA) caused by the intensified lipolytic activity, worsen insulin resistance through the impairment of insulin action; a mechanism known as lipotoxicity.

“Anti-diabetic treatment” for type 2 diabetes include:
Metformin. Generally, metformin is the first medication prescribed for type 2 diabetes. It works by improving the sensitivity of your body tissues to insulin so that your body uses insulin more effectively. Metformin also lowers glucose production in the liver. Metformin may not lower blood sugar enough on its own.
Dipeptidyl peptidase-4 (DPP-4) inhibitors. These medications also reduce blood sugar levels. They do not cause weight gain. Examples of these medications are sitagliptin, saxagliptin, vildagliptin and linagliptin.
Sulfonylureas. These medications help the body secrete more insulin. Examples of medications in this class include glyburide, glipizide, and glimepiride. Possible side effects include low blood sugar and weight gain.
Thiazolidinediones. Like metformin, these medications make the body's tissues more sensitive to insulin. This class of medications has been linked to weight gain and other more-serious side effects, such as an increased risk of heart failure and fractures. Because of these risks, these medications generally are not a first-choice treatment. Pioglitazone is an example of thiazolidinediones.
GLP-1 receptor agonists (GLP-1Ra). These medications slow digestion and help lower blood sugar levels. Their use is often associated with some weight loss. This class of medications is not recommended for use by itself. Exenatide, semaglutide and liraglutide are examples of GLP-1 receptor agonists.
SGLT2 inhibitors. These are the newest diabetes drugs on the market. They work by preventing the kidneys from reabsorbing sugar into the blood. Instead, the sugar is excreted in the urine. Examples include canagliflozin and dapagliflozin.
Insulin therapy. Some people who have type 2 diabetes need insulin therapy as well. In the past, insulin therapy was used as a last resort, but today it is often prescribed sooner because of its benefits.

As used herein the term “antibody” as referred to herein includes whole antibodies and any antigen binding fragment or single chains thereof (i.e., “antigen-binding portion” or “functional fragment”). A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, the term “functional fragment” of an antibody as used herein, refers to portions or fragments of an antibody that retain the ability to specifically bind to an antigen (e.g., a portion of ActRIIB). It has been shown that the antigen-binding function of an antibody can be performed by fragments of a full-length antibody. Examples of binding fragments encompassed within the term “functional fragment” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989), which consists of a V_{H} domain; and an isolated complementarity determining region (CDR). Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988; and Huston et al., 1988). Such single chain antibodies are also intended to be encompassed within the term “functional fragments” of an antibody. These antibody fragments are obtained using conventional techniques known to those of skill in the art, and the fragments are screened for utility in the same manner as are intact antibodies.

As used herein, the terms “monoclonal antibody” or “monoclonal antibody composition” as used herein refer to a preparation of antibody molecules of single molecular composition. A monoclonal antibody composition displays a single binding specificity and affinity for a particular epitope.

As used herein, the term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis as described in Knappik, et al. (2000). J Mol Biol 296, 57-86. A “human antibody” need not be produced by a human, human tissue or human cell. The human antibodies of the disclosure may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “recombinant human antibody”, as used herein, includes all human antibodies that are prepared, expressed, created or isolated by recombinant means, such as antibodies isolated from an animal (e.g. a mouse) that is transgenic or transchromosomal for human immunoglobulin genes or a hybridoma prepared therefrom, antibodies isolated from a host cell transformed to express the human antibody, e.g. from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, an antibody that “binds to ActRIIB polypeptide” is intended to refer to an antibody that binds to human ActRIIB polypeptide with a K_{D} of about 100 nM or less, about 10 nM or less, or about 1 nM or less. As used herein, the term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system or Solution Equilibrium Titration.

As used herein, the term “antagonist antibody” is intended to refer to an antibody that inhibits ActRIIB induced signaling activity in the presence of myostatin or of other ActRIIB ligands such as activins or GDF-11 and/or to an antibody that inhibits ActRIIA induced signaling activity in the presence of myostatin or of other ActRIIA ligands such as activins or GDF-11. Examples of an assay to detect this include inhibition of myostatin induced signaling (for instance by a Smad dependent reporter gene assay), inhibition of myostatin induced Smad phosphorylation (P-Smad ELISA) and inhibition of myostatin induced inhibition of skeletal muscle cell differentiation (for instance by a creatine kinase assay).

In some embodiments, the antibodies that binds to the ActRIIB polypeptide inhibit myostatin induced signaling as measured in a Smad dependent reporter gene assay at an IC₅₀ of about 10 nM or less, about 1 nM or less, or about 100 pM or less.

The term “assaying” is used to mean that a sample may be tested (either directly or indirectly) for either the presence or level of a given marker (e.g., hsCRP and/or hemoglobin). It will be understood that, in a situation where the level of a substance denotes a probability, then the level of such substance may be used to guide a therapeutic decision. For example, one may determine the level of hsCRP and/or hemoglobin in a patient by assaying for its presence by quantitative or relatively-quantitative means (e.g., levels relative to the levels in other samples).

### ActRII Antagonists

The various disclosed uses, methods, combinations and kits utilize an ActRII antagonist, e.g., ActRIIA and/or ActRIIB antagonist, e.g., anti-ActRII receptor antibody, e.g., the anti-ActRII receptor antibody bimagrumab. In some embodiments, the ActRII antagonist is an ActRIIA and/or ActRIIB antagonist, preferably an anti-ActRII receptor antibody or antigen-binding fragment thereof.

In one embodiment, the anti-ActRII receptor antibody or antigen-binding fragment thereof comprises at least one immunoglobulin heavy chain variable domain (VH) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the anti-ActRII receptor antibody or antigen-binding fragment thereof comprises at least one immunoglobulin light chain variable domain (VL′) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6.

In one embodiment, the anti-ActRII receptor antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the anti-ActRII receptor antibody or antigen-binding fragment thereof comprises: a) an immunoglobulin heavy chain variable domain (VH) comprising the amino acid sequence set forth as SEQ ID NO:10; b) an immunoglobulin light chain variable domain (VL) comprising the amino acid sequence set forth as SEQ ID NO:9; c) an immunoglobulin VH domain comprising the amino acid sequence set forth as SEQ ID NO:10 and an immunoglobulin VL domain comprising the amino acid sequence set forth as SEQ ID NO:9; d) an immunoglobulin VH domain comprising the hypervariable regions set forth as SEQ ID NO:1, SEQ ID NO:2, and SEQ ID NO:3; e) an immunoglobulin VL domain comprising the hypervariable regions set forth as SEQ ID NO:4, SEQ ID NO:5 and SEQ ID NO:6; or f) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO:5 and SEQ ID NO:6.

For ease of reference the amino acid sequences of the bimagrumab monoclonal antibody, is provided in Table 1.

TABLE 1
Sequence information for bimagrumab

| SEQ | | |
|---|---|---|
| ID | Description of | |
| NO: | sequence | Detailed amino acid or nucleotide sequences |
| 1 | Heavy chain | GYTFTSSYIN |
| | CDR1 | |
| 2 | Heavy chain | TINPVSGSTSYAQKFQG |
| | CDR2 | |
| 3 | Heavy chain | GGWFDY |
| | CDR3 | |
| 4 | Light chain | TGTSSDVGSYNYVN |
| | CDR1′ | |
| 5 | Light chain | LMIYGVSKRPS |
| | CDR2′ | |
| 6 | Light chain | GTFAGGSYYG |
| | CDR3′ | |
| 7 | Light chain (LC) | QSALTQPASVSGSPGQSITISCTGTSSDVGSYNYVNWYQQHPGKAPKL |
| | | MIYGVSKRPSGVSNRFSGSKSGNTASLTISGLQAEDEADYYCGTFAGG |
| | | SYYGVFGGGTKLTVLGQPKAAPSVTLFPPSSEELQANKATLVCLISDFY |
| | | PGAVTVAWKADSSPVKAGVETTTPSKQSNNKYAASSYLSLTPEQWKS |
| | | HRSYSCQVTHEGSTVEKTVAPTECS |
| 8 | Heavy chain | QVQLVQSGAEVKKPGASVKVSCKASGYTFTSSYINVVVRQAPGQGLEW |
| | (HC) | MGTINPVSGSTSYAQKFQGRVTMTRDTSISTAYMELSRLRSDDTAVYY |
| | | CARGGWFDYWGQGTLVTVSSASTKGPSVFPLAPSSKSTSGGTAALGC |
| | | LVKDYFPEPVTVSWNSGALTSGVHTFPAVLQSSGLYSLSSWTVPSSSL |
| | | GTQTYICNVNHKPSNTKVDKRVEPKSCDKTHTCPPCPAPEAAGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSREEMTKNQVSLTCLVKGFYPSDIAVEWESNGQ |
| | | PENNYKTTPPVLDSDGSFFLYSKLTVDKSRWQQGNVFSCSVMHEALH |
| | | NHYTQKSLSLSPGK |
| 9 | Variable Light | QSALTQPASVSGSPGQSITISCTGTSSDVGSYNYVNWYQQHPGKAPKL |
| | chain (VL) | MIYGVSKRPSGVSNRFSGSKSGNTASLTISGLQAEDEADYYCGTFAGG |
| | | SYYGVFGGGTKLTVL |
| 10 | Variable heavy | QVQLVQSGAEVKKPGASVKVSCKASGYTFTSSYINVVVRQAPGQGLEW |
| | chain (VH) | MGTINPVSGSTSYAQKFQGRVTMTRDTSISTAYMELSRLRSDDTAVYY |
| | | CARGGWFDYWGQGTLVTVSS |

In preferred embodiments, constant region domains also comprise suitable human constant region domains, for instance as described in “Sequences of Proteins of Immunological Interest”, Kabat E. A. et al, US Department of Health and Human Services, Public Health Service, National Institute of Health.

In some embodiments, anti-ActRII receptor antibody or antigen-binding fragment thereof comprises the light chain of SEQ ID NO:7. In other embodiments, the anti-ActRII receptor antibody or antigen-binding fragment thereof comprises the heavy chain of SEQ ID NO:8. In other embodiments, the anti-ActRII receptor antibody or antigen-binding fragment thereof comprises the light chain of SEQ ID NO:7 and the heavy chain of SEQ ID NO:8. In some embodiments, the anti-ActRII receptor antibody or antigen-binding fragment thereof comprises the three CDRs of SEQ ID NO:7. In other embodiments, anti-ActRII receptor antibody or antigen-binding fragment thereof comprises the three CDRs of SEQ ID NO:8. In other embodiments, the anti-ActRII receptor antibody or antigen-binding fragment thereof comprises the three CDRs of SEQ ID NO:7 and the three CDRs of SEQ ID NO:8. CDRs of SEQ ID NO:7 and SEQ ID NO:8 may be found in Table 1.

In one embodiment, the anti-ActRII receptor antibody or antigen-binding fragment thereof (e.g., bimagrumab) is selected from a human anti-ActRII receptor antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1′, CDR2′, and CDR3′ and the constant part or fragment thereof of a human light chain, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the anti-ActRII receptor antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an anti-ActRII receptor antibody or antigen-binding fragment thereof as used in the disclosed methods may comprise a derivative of the anti-ActRII receptor antibodies set forth herein by sequence (e.g., pegylated variants, glycosylation variants, affinity-maturation variants, etc.). Alternatively, the V_{H} or V_{L} domain of an anti-ActRII receptor antibody or antigen-binding fragment thereof used in the disclosed methods may have V_{H} or V_{L} domains that are substantially identical to the V_{H} or V_{L} domains set forth herein (e.g., those set forth in SEQ ID NO:10 and 9). A human anti-ActRII receptor antibody disclosed herein may comprise a heavy chain that is substantially identical to that set forth as SEQ ID NO:8 and/or a light chain that is substantially identical to that set forth as SEQ ID NO:7. A human anti-ActRII receptor antibody disclosed herein may comprise a heavy chain that comprises SEQ ID NO:8 and a light chain that comprises SEQ ID NO:7. A human anti-ActRII receptor antibody disclosed herein may comprise: a) one heavy chain which comprises a variable domain having an amino acid sequence substantially identical to that shown in SEQ ID NO:10 and the constant part of a human heavy chain; and b) one light chain which comprises a variable domain having an amino acid sequence substantially identical to that shown in SEQ ID NO:9 and the constant part of a human light chain.

Alternatively, an anti-ActRII receptor antibody or antigen-binding fragment thereof used in the disclosed methods may be an amino acid sequence variant of the reference anti-ActRII receptor antibodies set forth herein. The disclosure also includes anti-ActRII receptor antibodies or antigen-binding fragments thereof (e.g., bimagrumab) in which one or more of the amino acid residues of the VH or VL domain of bimagrumab, typically only a few (e.g., 1-10), are changed; for instance by mutation, e.g., site directed mutagenesis of the corresponding DNA sequences.

In some embodiments, the anti-ActRII antibody, e.g., bimagrumab, binds to an epitope of human ActRII receptor comprising WLDDFN (SEQ ID NO:11). In some embodiments, the anti-ActRII antibodies or antigen-binding fragments thereof, e.g., bimagrumab, bind to an epitope of human ActRII receptor comprising GCWLDDFNC (SEQ ID NO:12). In some embodiments, the anti-ActRII antibody, e.g., bimagrumab, binds to an epitope of human ActRII receptor comprising KGCWLDDFNCY (SEQ ID NO:13). In some embodiments, the anti-ActRII antibody, e.g., bimagrumab, binds to an epitope of human ActRII receptor comprising EQDKR (SEQ ID NO:14). In some embodiments, the anti-ActRII antibody, e.g., bimagrumab, binds to an epitope of human ActRII receptor comprising CEGEQDKRLHCYASW (SEQ ID NO:15). In some embodiments, the anti-ActRII antibody, e.g., bimagrumab, binds to an epitope of human ActRII receptor comprising WLDDFN (SEQ ID NO:11), CEGEQDKRLHCYASW (SEQ ID NO:15) and GCWLDDFNC (SEQ ID NO:12). In some embodiments, the anti-ActRII antibody, e.g., bimagrumab, binds to an epitope of human ActRII receptor comprising WLDDFN (SEQ ID NO:11) and CEGEQDKRLHCYASW (SEQ ID NO:15). In some embodiments, the anti-ActRII antibody, e.g., bimagrumab, binds to an epitope of human ActRII receptor comprising WLDDFN (SEQ ID NO:11) and EQDKR (SEQ ID NO:14). In some embodiments, the ActRII antibody has a KD of about 2-10 pM to human ActRIIB and a KD of about 100-600 pM to human ActRIIA (e.g., as determined by a Biacore® assay).

In a particularly preferred embodiment of any of the disclosed uses, methods, combinations and kits, the ActRII antagonist is bimagrumab. Bimagrumab has been described in WO2010/125003, which is hereby incorporated by reference in its entirety.

Bimagrumab, the pharmaceutically active compound used in accordance with the present disclosure, is a fully human, monoclonal antibody (modified IgG1, 234-235-Ala-Ala, A2) developed to bind competitively to activin receptor type II (ActRII) with greater affinity than its natural ligands, including myostatin and activin. Bimagrumab is cross-reactive with human and mouse ActRIIA and ActRIIB and effective on human, cynomolgus, mouse and rat skeletal muscle cells. Bimagrumab binds with extremely high affinity (KD 1.7±0.3 pM) to human ActRIIB and with relatively lower affinity to human ActRIIA (KD 434±25 pM).

Bimagrumab comprises an antigen binding site comprising at least one immunoglobulin heavy chain variable domain (V_{H}) which comprises in sequence hypervariable regions CDR1 of SEQ ID NO: 1, CDR2 of SEQ ID NO: 2 and CDR3 of SEQ ID NO: 3. The use of antibodies having 1, 2 or 3 residues changed from any of the sequences of CDR1, CDR2 and/or CDR3 of the heavy chain is also comprised within the scope of the disclosure.

Bimagrumab also comprises antigen binding site comprising at least one immunoglobulin light chain variable domain (V_{L}) which comprises in sequence hypervariable regions CDR1′ of SEQ ID NO: 4, CDR2′ of SEQ ID NO: 5 and CDR3′ of SEQ ID NO: 6 or CDR equivalents thereof.

The use of antibodies having 1, 2 or 3 residues changed from any of the sequences of CDR1′, CDR2′ and/or CDR3′ of the light chain is also comprised within the scope of the disclosure.

According to the disclosure the use of antibodies having 95% identity amino acid sequence with the light chain and/or the heavy chain of bimagrumab are also comprised.

Sequence listing for bimagrumab is provided herein.

Other preferred ActRII antagonists for use in the disclosed uses, methods, combinations and kits are those set forth in U.S. Pat. No. 7,893,213, WO2017147182, WO2016069234, WO2006012627, WO9956768, WO2002010214, WO2006012627, which are incorporated by reference herein in their entirety.

### Uses and Methods

The present disclosure arose in part from the analysis of the data generated from a clinical trial with ClinicalTrials.gov Identifier: NCT03005288 and as disclosed in WO2018/116201 (the contents of which are hereby incorporated by reference in their entirety), a randomized, subject- and investigator-blinded, placebo controlled study to assess the safety, pharmacokinetics and efficacy of intravenous bimagrumab in overweight and obese patients with type 2 diabetes. 75 patients were enrolled with type 2 diabetes with HbA1c between 6.5% and 10% and a Body Mass Index of 28 to 40 kg/m² at screening. Bimagrumab was administered every four weeks at a dose of 10 mg/kg with a maximum dose of 1200 mg intravenously for 12 doses and compared to placebo.

The inventors have now found that treatment with an anti-ActRII antibody, e.g., bimagrumab, significantly reduces hepatic fat fraction. Accordingly, activin receptor type II (ActRII) antagonists, e.g., molecules capable of antagonizing the binding of activins, growth differentiation factors (GDFs), bone morphogenetic proteins (BMPs) and/or myostatin to the human ActRII receptor, preferably an antagonist antibody to ActRIIA and/or ActRIIB, most preferably bimagrumab can be used according to the present disclosure to prevent or treat liver disease or disorder, in particular a liver disease or disorder associated with increased hepatic fat e.g., in associated with hepatic steatosis.

Various (enumerated) embodiments of the present disclosure are described herein. It will be recognized that features specified in each embodiment may be combined with other specified features to provide further embodiments of the present disclosure:

### Embodiments (a)

1a. An activin receptor type II (ActRII) antagonist for use in the treatment of liver disease or disorder in a subject in need thereof.
2a. An activin receptor type II (ActRII) antagonist for use in the prevention of liver disease or disorder in a subject in need thereof.
3a. An activin receptor type II (ActRII) antagonist for use in the treatment, stabilization or lessening the severity or progression of a non-alcoholic fatty liver disease (NAFLD), e.g. NASH, in a subject in need thereof, comprising administering the activin receptor type II (ActRII) antagonist at a therapeutically effective amount.
4a. An activin receptor type II (ActRII) antagonist for use in the slowing, arresting, or reducing the development of a chronic liver disease or disorder, e.g. NAFLD, non-alcoholic steatohepatitis (NASH), or liver fibrosis, in a subject in need thereof, comprising administering the activin receptor type II (ActRII) antagonist at a therapeutically effective amount.
5a. The ActRIIA/ActRIIB antagonist for use according to any one of embodiments 1a to 4a, wherein said subject has at least one condition selected from hepatic steatosis, lobular inflammation, and hepatocellular ballooning.
6a. The ActRIIA/ActRIIB antagonist for use according to any one of embodiments 1a to 5a, wherein said subject has hepatic steatosis.
7a. The ActRIIA/ActRIIB antagonist for use according to any one of embodiments 1a to 6a, wherein said liver disease or disorder is selected from non-alcoholic fatty liver disease (NAFLD) and non-alcoholic steatohepatitis (NASH).
8a. The ActRIIA/ActRIIB antagonist for use according to any one of embodiments 1a to 7a, wherein said liver disease or disorder is steatohepatitis.
9a. The ActRIIA/ActRIIB antagonist for use according to any one of embodiments 1a to 8a, wherein said liver disease or disorder is liver fibrosis.
10a. The ActRIIA/ActRIIB antagonist for use according to any one of embodiments 1a to 9a, wherein said liver disease or disorder is non-alcoholic fatty liver disease (NAFLD).
11a. The ActRII antagonist for use according to any one of embodiments 1a to 10a, wherein said liver disease or disorder is non-alcoholic steatohepatitis (NASH).
12a. The ActRII antagonist for use according to any one of embodiments 1a to 11a, wherein said liver disease or disorder is non-alcoholic steatohepatitis (NASH), and wherein NASH is mild to moderate with fibrosis level F2-F3.
13a. The ActRIIA/ActRIIB antagonist for use according to any one of embodiments 1a to 12a, wherein administration of a therapeutically effective amount of the ActRIIA/ActRIIB antagonist to said subject reduces the hepatic fat fraction in said subject compared to the hepatic fat fraction in said subject prior to the administration of a therapeutically effective amount of the ActRIIA/ActRIIB antagonist.
14a. The ActRIIA/ActRIIB antagonist for use according to any one of embodiments 1a to 13a, wherein administration of a therapeutically effective amount of the ActRIIA/ActRIIB antagonist to said subject reduces steatohepatitis compared to steatohepatitis prior to the administration of the ActRII antagonist.
15a. The activin receptor type II (ActRII) antagonist for use according to any one of embodiments 1a to 14a, wherein administration of a therapeutically effective amount of said ActRIIA/ActRIIB antagonist resolves steatohepatitis.
16a. The activin receptor type II (ActRII) antagonist for use according to any one of Embodiments 1a to 15a, wherein administration of a therapeutically effective amount of said ActRIIA/ActRIIB antagonist improves liver fibrosis.
17a. The activin receptor type II (ActRII) antagonist for use according to any one of embodiments 1a to 16a, wherein administration of a therapeutically effective amount of said ActRIIA/ActRIIB antagonist resolves steatohepatitis and improves liver fibrosis.
18a. The ActRIIA/ActRIIB antagonist for use according to any one of embodiments 1a to 17a, wherein administration of a therapeutically effective amount of said ActRIIA/ActRIIB antagonist alleviates or reduces progression of NASH.
19a. The ActRIIA/ActRIIB antagonist for use according to any one of embodiments 1a to 18a, wherein administration of a therapeutically effective amount of said ActRIIA/ActRIIB antagonist improves liver fibrosis by at least one stage compared to the stage of liver fibrosis prior to the administration of said ActRIIA/ActRIIB antagonist.
20a. The ActRIIA/ActRIIB antagonist for use according to any one of embodiments 1a to 19a, wherein administration of a therapeutically effective amount of said ActRIIA/ActRIIB antagonist halts progression to F3 or F4 liver fibrosis.
21a. The ActRIIA/ActRIIB antagonist for use according to any one of embodiments 1a to 20a, wherein administration of a therapeutically effective amount of said ActRIIA/ActRIIB antagonist reduces NAFLD Activity Score (NAS) by at least 1 point, at least 2 points or at least 3 points.
22a. The ActRIIA/ActRIIB antagonist for use according to any one of embodiments 1a to 21a, wherein administration of a therapeutically effective amount of said ActRIIA/ActRIIB antagonist reduces at least one of hepatosteatosis, hepatic inflammation and hepatocellular ballooning by at least 1 NAS point.
23a. The ActRIIA/ActRIIB antagonist for use according to any one of embodiments 1a to 22a, wherein administration of a therapeutically effective amount of said ActRIIA/ActRIIB antagonist reduces at least two of steatosis, hepatic inflammation and hepatocellular ballooning, e.g. hepatosteatosis and hepatic inflammation, or hepatosteatosis and hepatocellular ballooning, or hepatocellular ballooning and hepatic inflammation by at least one NAS point.
24a. The ActRIIA/ActRIIB antagonist for use according to one any of embodiments 1a to 23a, wherein said subject is a diabetic subject, an obese subject, or a subject with metabolic syndrome or with another metabolic disorder.
25a. The ActRIIA/ActRIIB antagonist for use according to any one of embodiments 1a to 24a, wherein said subject has type 2 diabetes.
26a. The ActRIIA/ActRIIB antagonist for use according to any one of embodiments 1a to 25a, wherein said subject is concomitantly receiving standard of care treatment for Type 2 diabetes.
27a. The ActRIIA/ActRIIB antagonist for use according to embodiment 26a, wherein the standard of care treatment is selected from metformin, DPP4 inhibitor, metformin/DPP4 inhibitor, sulfonylureas, thiazolidinediones, GLP-1 receptor agonists, SGLT2 inhibitors, insulin therapy.
28a. The ActRIIA/ActRIIB antagonist for use according to any of embodiments 1a to 27a, wherein said subject has HbA1c of 6.5% to 10%.
29a. The ActRIIA/ActRIIB antagonist for use according to any one of embodiments 1a to 28a, wherein said subject is obese or overweight or of normal BMI.
30a. The activin receptor type II (ActRII) antagonist for use according to any one of embodiments 1a to 29a, wherein the activin receptor type II (ActRII) antagonist is an ActRIIA and/or ActRIIB antagonist.
31a. The activin receptor type II (ActRII) antagonist for use according to any one of embodiments 1a to 30a, wherein the activin receptor type II (ActRII) antagonist is an ActRIIA and ActRIIB antagonist.
32a. The ActRIIA/ActRIIB antagonist for use according to any one of embodiments 1a to 31a, wherein the ActRIIA/ActRIIB antagonist is an anti-ActRII antibody or functional fragment thereof.
32a. The ActRIIA/ActRIIB antibody according to embodiment 31a, wherein said antibody or functional fragment thereof has a Kd of less than 1 nM for ActRIIA and less than 20 pM for ActRIIB.
33a. The ActRIIA/ActRIIB-binding antibody or functional fragment thereof according to embodiment
32a, wherein said ActRIIA/ActRIIB-binding antibody is selected from the group comprising:
a) an antibody comprising the three CDRs of SEQ ID NO:1, SEQ ID NO:2, SEQ ID NO:3;
b) an antibody comprising the three CDRs of SEQ ID NO:4, SEQ ID NO:5, SEQ ID NO:6;
c) an antibody comprising the three CDRs of SEQ ID NO:1, SEQ ID NO:2, SEQ ID NO:3 and the three CDRs of SEQ ID NO:4, SEQ ID NO:5, SEQ ID NO:6;
d) an ActRIIA/ActRIIB-binding antibody comprising a HC domain comprising SEQ ID NO:8;
e) an ActRIIA/ActRIIB-binding antibody comprising a LC domain comprising SEQ ID NO:7;
f) an ActRIIA/ActRIIB-binding antibody comprising a HC domain comprising SEQ ID NO:8 and a LC domain comprising SEQ ID NO:7,
g) an ActRIIA/ActRIIB-binding antibody comprising a VL domain comprising SEQ ID NO:9,
h) an ActRIIA/ActRIIB-binding antibody comprising a VH domain comprising SEQ ID NO:10,
i) an ActRIIA/ActRIIB-binding antibody comprising a VL domain comprising SEQ ID NO:9 and a VH domain comprising SEQ ID NO:10,
j) an antibody capable of binding to each of the following epitopes of ActRIIB:
   (i) WLDDFN (SEQ ID NO:11) and
   (ii) CEGEQDKRLHCYASW (SEQ ID NO:15).
k) an antibody capable of binding to each of the following epitopes of ActRIIB:
   (i) WLDDFN (SEQ ID NO:11)
   (ii) CEGEQDKRLHCYASW (SEQ ID NO:15) and
   (iii) GCWLDDFNC (SEQ ID NO:12).
j) an antibody, which is:
   (i) capable of binding to an epitope consisting of WLDDFN (SEQ ID NO:11) and
   (ii) capable of binding to an epitope consisting of CEGEQDKRLHCYASW (SEQ ID NO:15).
      34a. The ActRIIA/ActRIIB-binding antibody or functional fragment thereof according to embodiment
      33a, wherein the 3 CDRs of SEQ ID NO:10 are set forth in SEQ ID NO: 1, 2, and 3, and wherein the 3 CDRs of SEQ ID NO:9 are set forth in SEQ ID NO:4, 5, and 6.
      35a. The ActRIIA/ActRIIB-binding antibody or functional fragment thereof according to any one of embodiments 33a or 34a, wherein the 3 CDRs of SEQ ID NO:8 are set forth in SEQ ID NO: 1, 2, and 3, and wherein the 3 CDRs of SEQ ID NO:7 are set forth in SEQ ID NO:4, 5, and 6.
      36a. The ActRIIA/ActRIIB antagonist for use according to any one of embodiments 1a to 35a, wherein the ActRIIA/ActRIIB antagonist is bimagrumab.
      37a. The ActRIIA/ActRIIB antagonist for use according to any of embodiments 1a to 36a, comprising administering about 3 mg/kg to about 10 mg/kg bimagrumab.
      38a. The ActRIIA/ActRIIB antagonist for use according to any of embodiments 1a to 37a, comprising administering about 3 mg/kg bimagrumab.
      39a. The ActRIIA/ActRIIB antagonist for use according to any of embodiments 1a to 38a, comprising administering about 4 mg/kg bimagrumab.
      40a. The ActRIIA/ActRIIB antagonist for use according to any of embodiments 1a to 39a, comprising administering about 5 mg/kg bimagrumab.
      41a. The ActRIIA/ActRIIB antagonist for use according to any of embodiments 1a to 40a, comprising administering about 6 mg/kg bimagrumab.
      42a. The ActRIIA/ActRIIB antagonist for use according to any of embodiments 1a to 41a, comprising administering about 7 mg/kg bimagrumab.
      43a. The ActRIIA/ActRIIB antagonist for use according to any of embodiments 1a to 42a, comprising administering about 8 mg/kg bimagrumab.
      44a. The ActRIIA/ActRIIB antagonist for use according to any of embodiments 1a to 43a, comprising administering about 9 mg/kg bimagrumab.
      45a. The ActRIIA/ActRIIB antagonist for use according to any of embodiments 1a to 44a, comprising administering about 10 mg/kg bimagrumab.
      46a. The ActRIIA/ActRIIB antagonist for use according to any one of embodiments 1a to 45a, wherein bimagrumab is administered every 4 weeks.
      47a. The ActRIIA/ActRIIB antagonist for use according to any one of embodiments 1a to 46a, wherein bimagrumab is administered every 4 weeks for at least 3 months, at least 6 months, at least 9 months or at least 12 months.
      48a. The ActRIIA/ActRIIB antagonist for use according to any one of embodiments 1a to 47a, comprising administering at least one further therapeutic agent.
      49a. The ActRIIA/ActRIIB antagonist for use according to embodiment 48a, comprising administering the ActRIIA/ActRIIB antagonist in combination with at least one further therapeutic agent for the treatment or prevention of liver disease.
      50a. The ActRIIA/ActRIIB antagonist for use according to embodiment 49a, wherein the at least one further therapeutic agent is FXR agonist (e.g., tropifexor, nidufexor, obeticholic acid (6α-ethyl-chenodeoxycholic acid), cilofexor (GS-9674, Px-102), TERN-101 (LY2562175), EYP001 (PXL007), EDP-305, AKN-083 (Allergan), INT-787 (Intercept), INT-767 (Intercept), AGN-242256 (Allergan), MET409 (Metacrine), Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, IVA337 (Inventive), CER-002 (Cerenis), GLP-1 agonist (e.g., exenatide, liraglutide, semaglutide, NC-101 (Naia Metabolic), G-49 (Astrazeneca), ZP2929 (BI/Zealand), PB-718 (Peg Bio), FGF agonist (e.g., pegbelfermin (ARX618), BMS-986171, NGM-282, NGM-313, YH25724, tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), FXR agonist (M480 (Metacrine), NTX-023-1 (Ardelyx), INV-33 (Innovimmune)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption) or any combination thereof.

### Embodiments (b)

1b. A method for the treatment of liver disease or disorder in a subject in need thereof, comprising administering to said subject a therapeutically effective amount of an activin receptor type II (ActRII) antagonist.
2b. A method for the prevention of liver disease or disorder in a subject in need thereof, comprising administering to said subject a therapeutically effective amount of an activin receptor type II (ActRII) antagonist.
3b. A method for the treatment, stabilization or lessening the severity or progression of a non-alcoholic fatty liver disease (NAFLD), e.g., NASH, in a subject in need thereof, comprising administering to said subject a therapeutically effective amount of an activin receptor type II (ActRII) antagonist.
4b. A method for slowing, arresting, or reducing the development of a chronic liver disease or disorder, e.g., NAFLD, non-alcoholic steatohepatitis (NASH), or liver fibrosis, in a subject in need thereof, comprising administering to said subject a therapeutically effective amount of an activin receptor type II (ActRII) antagonist.
5b. The method according to any one of embodiments 1 b to 4b, wherein said subject has at least one condition selected from hepatic steatosis, lobular inflammation, and hepatocellular ballooning.
6b. The method according to any one of embodiments 1 b to 5b, wherein said subject has hepatic steatosis.
7b. The method according to any one of embodiments 1 b to 6b, wherein said liver disease or disorder is selected from non-alcoholic fatty liver disease (NAFLD) and non-alcoholic steatohepatitis (NASH).
8b. The method according to any one of embodiments 1 b to 7b, wherein said liver disease or disorder is steatohepatitis.
9b. The method according to any one of embodiments 1 b to 8b, wherein said liver disease or disorder is liver fibrosis.
10b. The method according to any one of embodiments 1 b to 9b, wherein said liver disease or disorder is non-alcoholic fatty liver disease (NAFLD).
11b. The method according to any one of embodiments 1 b to 10b, wherein said liver disease or disorder is non-alcoholic steatohepatitis (NASH).
12b. The method according to any one of embodiments 1 b to 11 b, wherein said liver disease or disorder is non-alcoholic steatohepatitis (NASH), and wherein NASH is mild to moderate with fibrosis level F2-F3.
13b. The method according to any one of embodiments 1 b to 12b, wherein administration of a therapeutically effective amount of the ActRIIA/ActRIIB antagonist to said subject reduces the hepatic fat fraction in said subject compared to the hepatic fat fraction in said subject prior to the administration of a therapeutically effective amount of the ActRIIA/ActRIIB antagonist.
14b. The method according to any one of embodiments 1 b to 13b, wherein administration of a therapeutically effective amount of the ActRIIA/ActRIIB antagonist to said subject reduces steatohepatitis compared to steatohepatitis prior to the administration of the ActRII antagonist.
15b. The method according to any one of embodiments 1 b to 14b, wherein administration of a therapeutically effective amount of said ActRIIA/ActRIIB antagonist resolves steatohepatitis.
16b. The method according to any one of Embodiments 1 b to 15b, wherein administration of a therapeutically effective amount of said ActRIIA/ActRIIB antagonist improves liver fibrosis.
17b. The method according to any one of embodiments 1 b to 16b, wherein administration of a therapeutically effective amount of said ActRIIA/ActRIIB antagonist resolves steatohepatitis and improves liver fibrosis.
18b. The method according to any one of embodiments 1 b to 17b, wherein administration of a therapeutically effective amount of said ActRIIA/ActRIIB antagonist alleviates or reduces progression of NASH.
19b. The method according to any one of embodiments 1 b to 18b, wherein administration of a therapeutically effective amount of said ActRIIA/ActRIIB antagonist improves liver fibrosis by at least one stage compared to the stage of liver fibrosis prior to the administration of said ActRIIA/ActRIIB antagonist.
20b. The method according to any one of embodiments 1 b to 19b, wherein administration of a therapeutically effective amount of said ActRIIA/ActRIIB antagonist halts progression to F3 or F4 liver fibrosis.
21b. The method according to any one of embodiments 1 b to 20b, wherein administration of a therapeutically effective amount of said ActRIIA/ActRIIB antagonist reduces NAFLD Activity Score (NAS) by at least 1 point, at least 2 points or at least 3 points.
22b. The method according to any one of embodiments 1 b to 21b, wherein administration of a therapeutically effective amount of said ActRIIA/ActRIIB antagonist reduces at least one of hepatosteatosis, hepatic inflammation and hepatocellular ballooning by at least 1 NAS point.
23b. The method according to any one of embodiments 1 b to 22b, wherein administration of a therapeutically effective amount of said ActRIIA/ActRIIB antagonist reduces at least two of steatosis, hepatic inflammation and hepatocellular ballooning, e.g. hepatosteatosis and hepatic inflammation, or hepatosteatosis and hepatocellular ballooning, or hepatocellular ballooning and hepatic inflammation by at least 1 NAS point.
24b. The method according to one any of embodiments 1 b to 23b, wherein said subject is a diabetic subject, an obese subject, or a subject with metabolic syndrome or with another metabolic disorder.
25b. The method according to any one of embodiments 1 b to 24b, wherein said subject has type 2 diabetes.
26b. The method according to any one of embodiments 1 b to 25b, wherein said subject is concomitantly receiving standard of care treatment for Type 2 diabetes.
27b. The method according to embodiment 26b, wherein the standard of care treatment is selected from metformin, DPP4 inhibitor, metformin/DPP4 inhibitor, sulfonylureas, thiazolidinediones, GLP-1 receptor agonists, SGLT2 inhibitors, insulin therapy.
28b. The method according to any of embodiments 1 b to 27b, wherein said subject has HbA1c of 6.5% to 10%.
29b. The method according to any one of embodiments 1 b to 28b, wherein said subject is obese or overweight or of normal BMI.
30b. The method according to any one of embodiments 1 b to 29b, wherein the activin receptor type II (ActRII) antagonist is an ActRIIA and/or ActRIIB antagonist.
31b. The method according to any one of embodiments 1 b to 30a, wherein the activin receptor type II (ActRII) antagonist is an ActRIIA and ActRIIB antagonist.
32b. The method according to any one of embodiments 1 b to 31b, wherein the ActRIIA/ActRIIB antagonist is an anti-ActRII antibody or functional fragment thereof.
32b. The method according to embodiment 31b, wherein said antibody or functional fragment thereof has a Kd of less than 1 nM for ActRIIA and less than 20 pM for ActRIIB.
33b. The method according to embodiment 32b, wherein said ActRIIA/ActRIIB-binding antibody is selected from the group comprising:
a) an antibody comprising the three CDRs of SEQ ID NO:1, SEQ ID NO:2, SEQ ID NO:3;
b) an antibody comprising the three CDRs of SEQ ID NO:4, SEQ ID NO:5, SEQ ID NO:6;
c) an antibody comprising the three CDRs of SEQ ID NO:1, SEQ ID NO:2, SEQ ID NO:3 and the three CDRs of SEQ ID NO:4, SEQ ID NO:5, SEQ ID NO:6;
d) an ActRIIA/ActRIIB-binding antibody comprising a HC domain comprising SEQ ID NO:8;
e) an ActRIIA/ActRIIB-binding antibody comprising a LC domain comprising SEQ ID NO:7;
f) an ActRIIA/ActRIIB-binding antibody comprising a HC domain comprising SEQ ID NO:8 and a LC domain comprising SEQ ID NO:7,
g) an ActRIIA/ActRIIB-binding antibody comprising a VL domain comprising SEQ ID NO:9,
h) an ActRIIA/ActRIIB-binding antibody comprising a VH domain comprising SEQ ID NO:10,
i) an ActRIIA/ActRIIB-binding antibody comprising a VL domain comprising SEQ ID NO:9 and a VH domain comprising SEQ ID NO:10,
j) an antibody capable of binding to each of the following epitopes of ActRIIB:

(i)
(SEQ ID NO: 11)
WLDDFN
and
(ii)
(SEQ ID NO: 15)
CEGEQDKRLHCYASW.

k) an antibody capable of binding to each of the following epitopes of ActRIIB:

(i)
(SEQ ID NO: 11)
WLDDFN
(ii)
(SEQ ID NO: 15)
CEGEQDKRLHCYASW
and
(iii)
(SEQ ID NO: 12)
GCWLDDFNC.

j) an antibody, which is:

(SEQ ID NO: 11)
(i) capable of binding to an epitope consisting
of WLDDFN
and
(SEQ ID NO: 15)
(ii) capable of binding to an epitope consisting
of CEGEQDKRLHCYASW.

34b. The ActRIIA/ActRIIB-binding antibody or functional fragment thereof according to embodiment
33a, wherein the 3 CDRs of SEQ ID NO:10 are set forth in SEQ ID NO: 1, 2, and 3, and wherein the 3 CDRs of SEQ ID NO:9 are set forth in SEQ ID NO:4, 5, and 6.
35b. The method according to any one of embodiments 33b or 34b, wherein the 3 CDRs of SEQ ID NO:8 are set forth in SEQ ID NO: 1, 2, and 3, and wherein the 3 CDRs of SEQ ID NO:7 are set forth in SEQ ID NO:4, 5, and 6.
36b. The method according to any one of embodiments 1 b to 35b, wherein the ActRIIA/ActRIIB antagonist is bimagrumab.
37b. The method according to any of embodiments 1 b to 36b, comprising administering about 3 mg/kg to about 10 mg/kg bimagrumab.
38b. The method according to any of embodiments 1 b to 37b, comprising administering about 3 mg/kg bimagrumab.
39b. The method according to any of embodiments 1 b to 38b, comprising administering about 4 mg/kg bimagrumab.
40b. The method according to any of embodiments 1 b to 39b, comprising administering about 5 mg/kg bimagrumab.
41b. The method according to any of embodiments 1 b to 40b, comprising administering about 6 mg/kg bimagrumab.
42b. The method according to any of embodiments 1 b to 41b, comprising administering about 7 mg/kg bimagrumab.
43b. The method according to any of embodiments 1 b to 42b, comprising administering about 8 mg/kg bimagrumab.
44b. The method according to any of embodiments 1 b to 43b, comprising administering about 9 mg/kg bimagrumab.
45b. The method according to any of embodiments 1 b to 44b, comprising administering about 10 mg/kg bimagrumab.
46b. The method according to any one of embodiments 1 b to 45b, wherein bimagrumab is administered every 4 weeks.
47b. The method according to any one of embodiments 1 b to 46b, wherein bimagrumab is administered every 4 weeks for at least 3 months, at least 6 months, at least 9 months or at least 12 months.
48b. The method according to any one of embodiments 1 b to 47b, comprising administering at least one further therapeutic agent.
49b. The method according to embodiment 48b, comprising administering the ActRIIA/ActRIIB antagonist in combination with at least one further therapeutic agent for the treatment or prevention of liver disease.
50b. The method according to embodiment 49b, wherein the at least one further therapeutic agent is FXR agonist (e.g., tropifexor, nidufexor, obeticholic acid (6α-ethyl-chenodeoxycholic acid), cilofexor (GS-9674, Px-102), TERN-101 (LY2562175), EYP001 (PXL007), EDP-305, AKN-083 (Allergan), INT-787 (Intercept), INT-767 (Intercept), AGN-242256 (Allergan), MET409 (Metacrine), Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, IVA337 (Inventive), CER-002 (Cerenis), GLP-1 agonist (e.g., exenatide, liraglutide, semaglutide, NC-101 (Naia Metabolic), G-49 (Astrazeneca), ZP2929 (BI/Zealand), PB-718 (Peg Bio), FGF agonist (e.g., pegbelfermin (ARX618), BMS-986171, NGM-282, NGM-313, YH25724, tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), FXR agonist (M480 (Metacrine), NTX-023-1 (Ardelyx), INV-33 (Innovimmune)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption) or any combination thereof.

### Embodiments (c)

1c. A pharmaceutical composition comprising an ActRIIA/ActRIIB antagonist, suitably bimagrumab, and at least one pharmaceutically acceptable excipient for use in the treatment or prevention of liver disease or disorder, in a subject in need thereof, comprising administering a therapeutically effective amount of the ActRIIA/ActRIIB antagonist, suitably bimagrumab.
2c. A pharmaceutical composition comprising an ActRIIA/ActRIIB antagonist, suitably bimagrumab, for use according to any one of Embodiments 1a to 50a, and at least one pharmaceutically acceptable excipient.

### Embodiments (d)

1d. Use of an ActRIIA/ActRIIB antagonist as defined in any one of embodiments 1a to 50a, for the manufacture of a medicament for the treatment of liver disease or disorder.
2d. Use of an ActRIIA/ActRIIB antagonist as defined in any one of embodiments 1a to 50a, for the manufacture of a medicament for the prevention of liver disease or disorder.
3d. Use of bimagrumab in the manufacture of a medicament for treating or preventing liver disease or disorder.
4d. The use of bimagrumab according to embodiment 3d, wherein said liver disease or disorder non-alcoholic fatty liver disease (NAFLD), non-alcoholic steatohepatitis (NASH), or liver fibrosis.
5d. The use of bimagrumab according to embodiment 4d, wherein the liver disease or disorder is NASH.

### Embodiments (e)

1e. Use of a pharmaceutical composition comprising an ActRIIA/ActRIIB antagonist according to any one of embodiment 1a to 50a and at least one pharmaceutically acceptable carrier, for the manufacture of a medicament for the treatment or prevention of liver disease or disorder.

An ActRIIA/ActRIIB antagonist, e.g., bimagrumab, a method, a pharmaceutical composition, or a use, according to any one of above listed Embodiments, wherein NAFLD is characterized by a NAFLD activity score (NAS) greater than or equal to 1, greater than or equal to 2, greater than or equal to 3 or greater than or equal to 4.

An ActRIIA/ActRIIB antagonist, a method, a pharmaceutical composition, or a use, according to any one of above listed embodiments, wherein NASH is confirmed based on liver biopsy (also called biopsy-proven NASH) and NASH is mild to moderate with fibrosis level F2-F3.

An ActRIIA/ActRIIB antagonist, or a method, a pharmaceutical composition, or a use, according to any one of the above listed embodiments, wherein presence of NASH has been demonstrated by:
i) Histologic evidence of NASH based on liver biopsy obtained 2 years or less before treatment with an ActRIIA/ActRIIB antagonist according to any one of the above Embodiments, with a diagnosis consistent with NASH, fibrosis level F1, F2, F3 or F4, no diagnosis of alternative chronic liver diseases, or
ii) Phenotypic diagnosis of NASH, or
iii) Noninvasive, disease-specific biomarkers.

An ActRIIA/ActRIIB antagonist, e.g., bimagrumab, a method, a pharmaceutical composition, or a use, according to any one of above listed embodiments, wherein the liver disease is associated with imbalanced liver lipid metabolism and/or increased fat deposits.

In any one of the above described embodiments the levels of hepatic fat content are assessed by magnetic resonance imaging (MRI), more particularly by magnetic resonance imaging-estimated proton density fat fraction (MRI-PDFF).

In any of the embodiments described herein, administration of a therapeutically acceptable amount of the the ActRIIA/ActRIIB antagonist, e.g., bimagrumab, results in a reduction in hepatic fat fraction in a patient. For example, in any one of the above described embodiments, treatment with an ActRII antagonist, preferably an ActRIIA and/or ActRIIB antagonist, and more preferably an anti-ActRII receptor antibody, most preferably bimagrumab, results in a reduction in hepatic fat fraction in the patient compared to the patient's hepatic fat fraction prior to treatment, optionally wherein hepatic fat fraction is as assessed by magnetic resonance imaging-estimated proton density fat fraction (MRI-PDFF).

In any one of the above described embodiments, treatment with an ActRII antagonist, preferably an ActRIIA and/or ActRIIB antagonist, and more preferably an anti-ActRII receptor antibody, most preferably bimagrumab, results in a 40% or greater (e.g., 41%, 42%, 43%, 44%, 45%, 46%, 47%, 48%, 49% 50%, 51%, 52%, 53%, 54%, 55%, 56%, 57%, 58%, 59%, 60%, 65%, 70%, 75% or greater) reduction in hepatic fat fraction in the patient compared to the patient's hepatic fat fraction prior to treatment, optionally wherein hepatic fat fraction is assessed by MRI-PDFF. In any one of the above described embodiments, the patient's hepatic fat fraction is reduced by at least 2 (e.g., at least 3, at least 4, at least 5, at least 6, at least 7, at least 8, at least 9, at least 10, at least 11 or at least 12) points following administration of an ActRII antagonist, preferably an ActRIIA and/or ActRIIB antagonist, and more preferably an anti-ActRII receptor antibody, most preferably bimagrumab, to the patient, compared to the patient's hepatic fat fraction prior to treatment, optionally wherein hepatic fat fraction is assessed by MRI-PDFF.

In some aspects, the ActRIIA/ActRIIB antagonist as defined herein, are provided for the treatment of a liver disease or disorder, e.g. a chronic liver disease or disorder, e.g. a disease or disorder selected from the group comprising cholestasis, intrahepatic cholestasis, estrogen-induced cholestasis, drug-induced cholestasis, cholestasis of pregnancy, parenteral nutrition-associated cholestasis, primary biliary cholangitis (PBC), primary sclerosing cholangitis (PSC), progressive familiar cholestasis (PFIC), non-alcoholic fatty liver disease (NAFLD), non-alcoholic steatohepatitis (NASH), steatohepatitis, drug-induced bile duct injury, gallstones, liver cirrhosis, alcohol-induced cirrhosis, cystic fibrosis-associated liver disease (CFLD), bile duct obstruction, cholelithiasis, liver fibrosis, dyslipidemia, portal hypertension, metabolic syndrome, hypercholesterolemia, progressive fibrosis of the liver caused by any of the diseases above and/or by infectious hepatitis, e.g., the liver disease or disorder is NAFLD, NASH, hepatic fibrosis, hepatosteatitis or hepatic steatosis.

In yet another aspect, a pharmaceutical composition is provided in the form of a unit dosage form comprising about 100 to about 200 mg/ml of bimagrumab, preferably about 100, about 105, about 110, about 115, about 120, about 125, about 130, about 135, about 140, about 145, about 150, about 155, about 160, about 165, about 170, about 175, about 180, about 185, about 190, about 195, about 200 mg/ml of bimagrumab of bimagrumab. Such unit dosage form compositions may be in a suitable form for intravenous administration. Such unit dosage form compositions may be in a suitable form for subcutaneous administration. Also these unit dosage form compositions are for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH), e.g. for use in treating phenotypic non-alcoholic steatohepatitis (NASH).

In yet another aspect, the ActRIIA/ActRIIB antagonist as defined herein is provided for preventing or delaying progression of a chronic liver disease or disorder to a more advanced stage or a more serious condition thereof, e.g. for preventing or delaying progression of a chronic liver disease or disorder selected from the group consisting of NAFLD, NASH, and hepatic fibrosis.

### Subjects

According to the disclosure, the subjects receiving the ActRIIA/ActRIIB antagonist as presently described can be affected or at risk of a liver disease or disorder, e.g., as hereinabove defined.

In some embodiments, the subject with liver disease or disorder is obese, overweight or of normal BMI. In one embodiment, the subject with liver disease or disorder has a body mass index (BMI) of 25 kg/m2 or greater. In another embodiment, the subject with liver disease or disorder has a BMI of 26 kg/m2 or greater. In another embodiment, the subject with liver disease or disorder has a BMI of 27 kg/m2 or greater. In another embodiment, the subject with liver disease or disorder has a BMI of 28 kg/m2 or greater. In another embodiment, the subject with liver disease or disorder has a BMI of 29 kg/m2 or greater. In one embodiment, the subject with liver disease or disorder is obese. In another embodiment, the subject with liver disease or disorder has a BMI of 30 kg/m2 or greater. In one embodiment, the subject with liver disease or disorder is overweight. In another embodiment, the subject with liver disease or disorder has a BMI of 25 and <30 kg/m2. In one embodiment, the subject with liver disease or disorder is of normal BMI. In another embodiment, the subject with liver disease or disorder has a BMI of less than 25 kg/m2.

In one embodiment, the subject with liver disease or disorder has hypertension and/or hypertriglyceridemia and/or low high-density lipoprotein (HDL).

In one embodiment, the subject with liver disease or disorder has type 2 diabetes and a BMI ≥30 kg/m² and at least one of hypertension, hypertriglyceridemia, and low HDL

### Combination Therapy

In practicing some of the methods of treatment or uses of the present disclosure, a therapeutically effective amount of an ActRII antagonist, e.g., the ActRIIA and/or ActRIIB antagonist, (e.g., the anti-ActRII receptor antibody or antigen-binding fragment thereof, e.g., bimagrumab) is administered to a patient, e.g., a mammal (e.g., a human). While it is understood that the disclosed methods provide for treatment of patients liver disease or disorder using an ActRII antagonist (e.g., bimagrumab), this does not preclude that, if the patient is to be ultimately treated with an ActRII antagonist, such ActRII antagonist therapy is necessarily a monotherapy. Indeed, if a patient is selected for treatment with an ActRII antagonist, then the ActRII antagonist (e.g., bimagrumab) may be administered in accordance with the methods or uses of the disclosure either alone or in combination with other agents and therapies for treating liver disease or disorder patients, e.g., in combination with at least one additional therapeutic agent. When co-administered with one or more additional therapeutic agent(s), an ActRII antagonist (e.g., bimagrumab) may be administered either simultaneously with the other agent, separately or sequentially. If administered sequentially, the attending physician will decide on the appropriate sequence of administering the ActRII antagonist (e.g., bimagrumab) in combination with other agents and the appropriate dosages for co-delivery. Accordingly, provided in one aspect of the disclosure, also provided is a pharmaceutical combination comprising (a) an ActRII antagonist, preferably an ActRIIA and/or ActRIIB antagonist, and more preferably an anti-ActRII receptor antibody, most preferably bimagrumab, and (b) at least one additional therapeutic agent.

Various therapies may be beneficially combined with the disclosed ActRII antagonist (e.g., bimagrumab) in the treatment or prevention of liver disease or disorder in a subject in need thereof. The at least one additional therapeutic agent may be FXR agonist, Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, IVA337 (Inventive), CER-002 (Cerenis), GLP-1 agonist (e.g., exenatide, liraglutide, semaglutide, NC-101 (Naia Metabolic), G-49 (Astrazeneca), ZP2929 (BI/Zealand), PB-718 (Peg Bio), FGF agonist (e.g., pegbelfermin (ARX618), BMS-986171, NGM-282, NGM-313, YH25724, tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), FXR agonist (M480 (Metacrine), NTX-023-1 (Ardelyx), INV-33 (Innovimmune)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption) or any combination thereof.

As used herein, a “FXR agonist”/“FXR agonists” refer to any agent that is capable of binding and activating farnesoid X receptor (FXR) which may be referred to as bile acid receptor (BAR) or NR1H4 (nuclear receptor subfamily 1, group H, member 4) receptor. FXR agonist may act as agonists or partial agonists of FXR. The agent may be e.g. a small molecule, an antibody or a protein, preferably a small molecule. The activity of a FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

The FXR agonist as used herein refers, for example, to compounds disclosed in: WO2016/096116, WO2016/127924, WO2017/218337, WO2018/024224, WO2018/075207, WO2018/133730, WO2018/190643, WO2018/214959, WO2016/096115, WO2017/118294, WO2017/218397, WO2018/059314, WO2018/085148, WO2019/007418, CN109053751, CN104513213, WO2017/128896, WO2017/189652, WO2017/189663, WO2017/189651, WO2017/201150, WO2017/201152, WO2017/201155, WO2018/067704, WO2018/081285, WO2018/039384, WO2015/138986, WO2017/078928, WO2016/081918, WO2016/103037, WO2017/143134.

The FXR agonist is preferably selected from: tropifexor, nidufexor, obeticholic acid (6α-ethyl-chenodeoxycholic acid), cilofexor (GS-9674, Px-102),

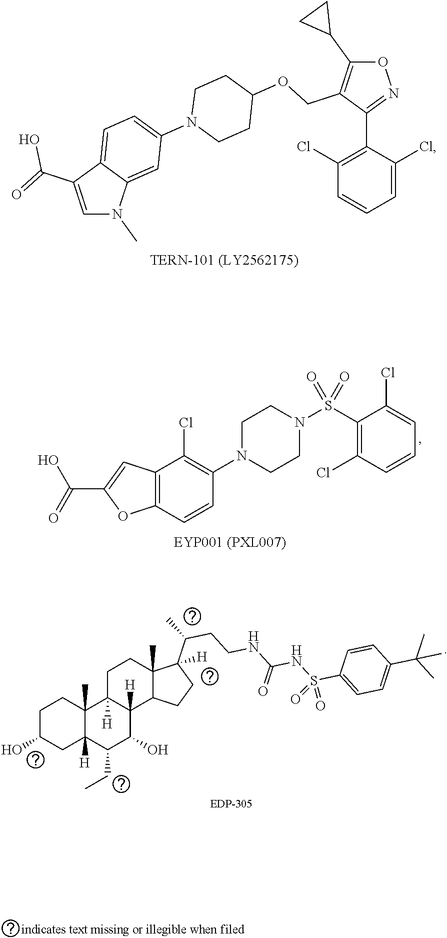

### Pharmaceutical Compositions

The ActRII antagonist, e.g., the ActRIIA and/or ActRIIB antagonist, e.g., the anti-ActRII receptor antibody or antigen-binding fragment thereof, e.g., bimagrumab, may be used as a pharmaceutical composition when combined with a pharmaceutically acceptable carrier. Such a composition may contain, in addition to an ActRII antagonist, carriers, various diluents, fillers, salts, buffers, stabilizers, solubilizers, and other materials known in the art. The characteristics of the carrier will depend on the route of administration. The pharmaceutical compositions for use in the disclosed methods may also contain at least one or more additional therapeutic agents for treatment of the particular targeted disorder. For example, a pharmaceutical composition may also include anti-diabetic agents or agents that aid weight loss or agents that are beneficial for the treatment of metabolic disorders or agents that may be used in the treatment or prevention of liver disease or disorder. Such additional factors and/or agents may be included in the pharmaceutical composition to produce a synergistic effect with the ActRII binding molecules, or to minimize side effects caused by the ActRII antagonists, e.g., ActRIIA and/or ActRIIB antagonist, (e.g., the anti-ActRII receptor antibody or antigen-binding fragment thereof, e.g., bimagrumab). In preferred embodiments, the pharmaceutical compositions for use in the disclosed methods comprise bimagrumab at 150 mg/ml.

Pharmaceutical compositions disclosed herein may be manufactured in a conventional manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, it may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution.

In some embodiments of the disclosed methods and uses, the ActRII antagonist, e.g., ActRII antibody, e.g., bimagrumab, is formulated as a lyophilizate. When a therapeutically effective amount of an ActRII antagonist, e.g., the ActRIIA and/or ActRIIB antagonist, (e.g., the anti-ActRII receptor antibody or antigen-binding fragment thereof, e.g., bimagrumab) is administered, the ActRII antagonist will be in the form of a pyrogen-free, parenterally acceptable solution. A pharmaceutical composition for intravenous) or subcutaneous injection may contain, in addition to the ActRII antagonist, an isotonic vehicle such as sodium chloride, Ringer's solution, dextrose, dextrose and sodium chloride, lactated Ringer's solution, or other vehicle as known in the art. The pharmaceutical composition of the disclosure can be formulated to be compatible with its intended route of administration (e.g., oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Dosing Regimen and Modes of Administration

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of sensitivity in individuals

Depending on the compound used, the targeted disease or disorder and the stage of such disease or disorder, the dosing regimen, i.e. administered doses and/or frequency of the pharmaceutical composition comprising the ActRII antagonist, e.g., the ActRIIA and/or ActRIIB antagonist, (e.g., the anti-ActRII receptor antibody or antigen-binding fragment thereof, e.g., bimagrumab), may vary. Depending on the compound used, the targeted disease or disorder and the stage of such disease or disorder, the dosing regimen, i.e. administered doses and/or frequency of the pharmaceutical combination comprising a) an ActRII antagonist, e.g., the ActRIIA and/or ActRIIB antagonist, (e.g., the anti-ActRII receptor antibody or antigen-binding fragment thereof, e.g., bimagrumab, and b) at least one further therapeutic agent, may vary.

For administration of the antibody comprising composition in the methods for treating liver disease or liver disorder or for use in the treatment or prevention of liver disease or liver disorder, the antibody dosage ranges from about 0.0001 to about 100 mg/kg, and more usually about 0.01 to about 30 mg/kg, of the subject's body weight. For example, dosages are about 3 mg/kg body weight, about 5 mg/kg body weight or about 10 mg/kg body weight within the ranges of about 3 to about 10 mg/kg, e.g., about 3, about 4, about 5, about 6, about 7, about 8, about 9, about 10 mg/kg body weight. Dosages are repeated as necessary and may be in the range from about once per week up to about once every 10 weeks, e.g. once every 4 weeks or once every 8 weeks.

### Kits

The disclosure also encompasses kits for use in the methods for treating or preventing liver disease or disorder, which may comprise an ActRII antagonist, e.g., the ActRIIA and/or ActRIIB antagonist, (e.g., the anti-ActRII receptor antibody or antigen-binding fragment thereof, e.g., bimagrumab) e.g., in liquid or lyophilized form or a pharmaceutical composition comprising such ActRII antagonist, e.g., bimagrumab. Additionally, such kits may comprise means for administering the ActRII antagonist (e.g., a syringe and vial, a prefilled syringe, a prefilled pen) and instructions for use. These kits may contain additional therapeutic agents (described supra), e.g., for delivery in combination with the enclosed ActRII antagonist, e.g., bimagrumab.

The phrase “means for administering” is used to indicate any available implement for systemically administering a drug top a patient, including, but not limited to, a pre-filled syringe, a vial and syringe, an injection pen, an autoinjector, an i.v. drip and bag, a pump, etc. With such items, a patient may self-administer the drug (i.e., administer the drug on their own behalf) or a physician may administer the drug.

Each component of the kit is usually enclosed within an individual container, and all of the various containers are within a single package along with instructions for use.

It is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

Other features, objects, and advantages of the disclosure will be apparent from the description and drawings, and from the claims.

### EXAMPLES

The following Examples illustrate the disclosure described above; they are not, however, intended to limit the scope of the disclosure in any way. Other variants of the disclosure will be readily apparent to one of ordinary skill in the art and are encompassed by the appended claims.

### Example 1 and 2: Pre-Clinical Study

Patients with NAFLD have been observed to have high expression of activin A and in patients with NASH, high levels of activin A were significantly related to the degree of hepatic fibrosis (Yndestad et al, Am J Gastroenterol. 2009 September; 104(9):2196-205). Several mouse models are available to study liver fibrosis, including the high fat diet-induced obesity NASH model, a long term (20 weeks) disease model relevant to treatment in NASH, which can be used to study the effect of treatment on NASH with fibrosis in a therapeutic treatment regimen, as well as the CCl₄-induced liver fibrosis model, a short term (4 weeks) chemically induced model, which can be applied to study the effect of treatment on fibrosis in a preventive treatment regimen.

### Example 1: DAX19 In Vivo 1—HF/NASH Diet Induced Obesity NASH Model

Adult male C57BL/6J mice were housed with ad libitum access to water and food. Mice were fed a HF/NASH diet (40 kcal % fat, 2% cholesterol, 40 kcal % carbohydrate, Research Diets, D09100301 or SSniff Special Diets, supplemented with a fructose-sucrose solution (42 g/L, 55% fructose and 45% sucrose by weight) in drinking water). Age-matched animals were maintained on regular chow (Normal Diet, ND, Kliba Nafag, 3892) and received tap water. Mice were subjected to HF/NASH diet for a total of 20 weeks. At week 8 of HF/NASH feeding, HF/NASH animals were randomized to treated and untreated groups according to body weight, total lean and fat masses, and liver fat measured by MRI. The study comprised three groups of mice: Group 1: Normal Diet/Water (n=7); Group 2: HF/NASH+Control antibody (SB-18-SN99, at 30 mg/kg) s.c., q7d (n=9) and Group 3: HF/NASH+CDD866 30 mg/kg, s.c., q7d (n=9). CDD866 is a chimeric murinized version of BYM338 (bimagrumab), where the human Fc region of the antibody has been replaced by a mouse Fc. Body weight was measured weekly, and fat and lean masses were measured at 0, 4, 7, 14 and 20 weeks of HF/NASH feeding using a mouse body composition nuclear magnetic resonance (NMR) analyzer and liver fat was assessed at 8, 12, 16 and 20 weeks of HF/NASH feeding using magnetic resonance imaging (MRI).

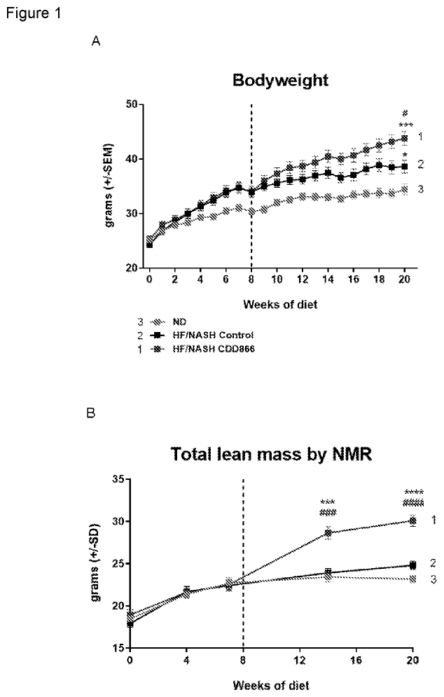

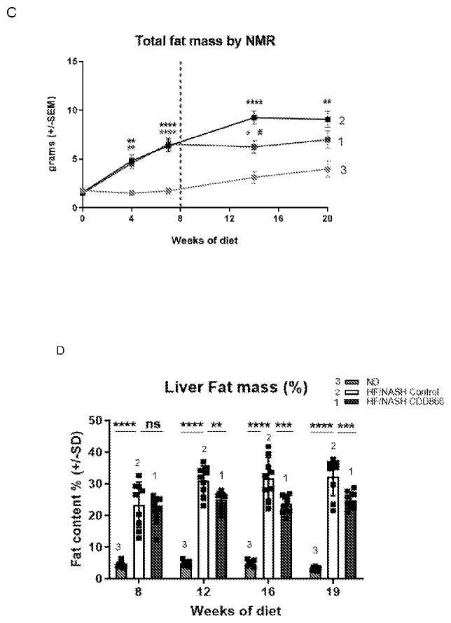

As shown in FIG. 1, increase in body weight (FIG. 1A) and lean mass (FIG. 1B) was observed upon CDD866 treatment. Total fat mass was decreased at week 14 of HF/NASH feeding (FIG. 1C).% liver fat decreased at all measured time points including reaching 20% and 24% decrease at weeks 12 and 20 of HF/NASH, respectively (FIG. 1D).

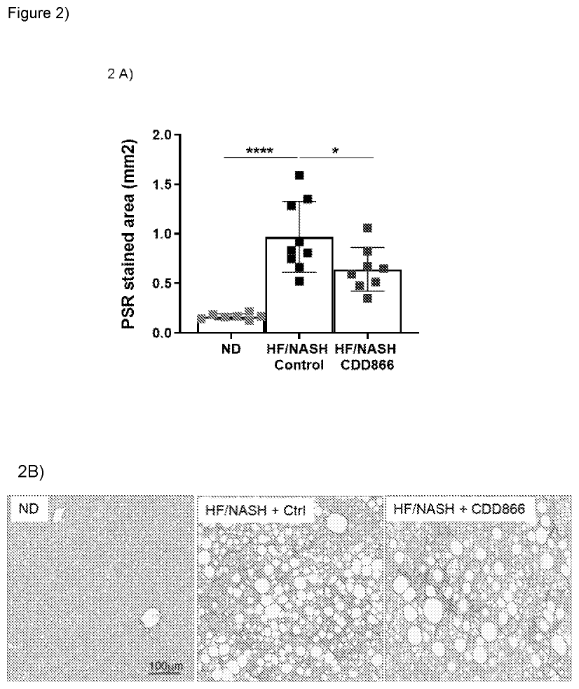

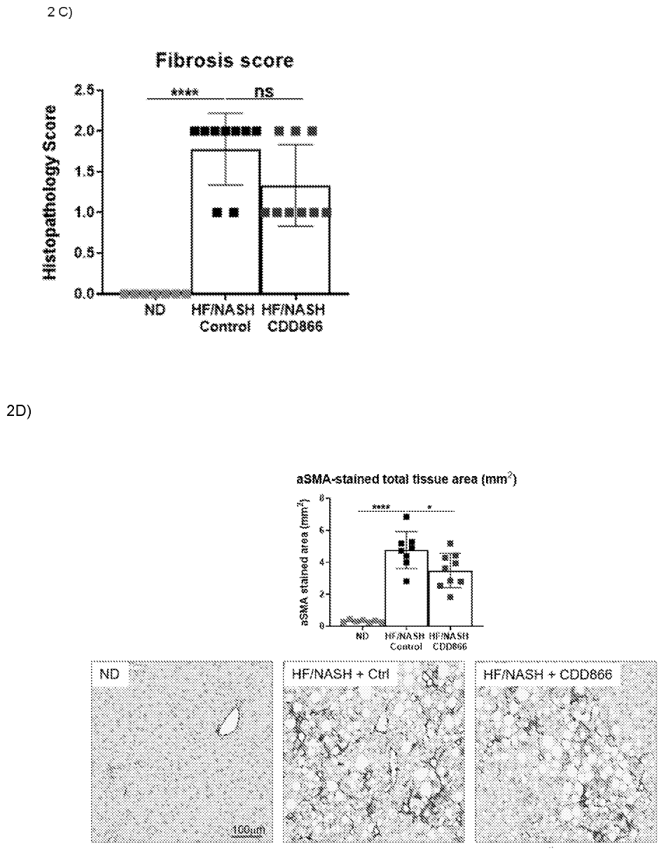

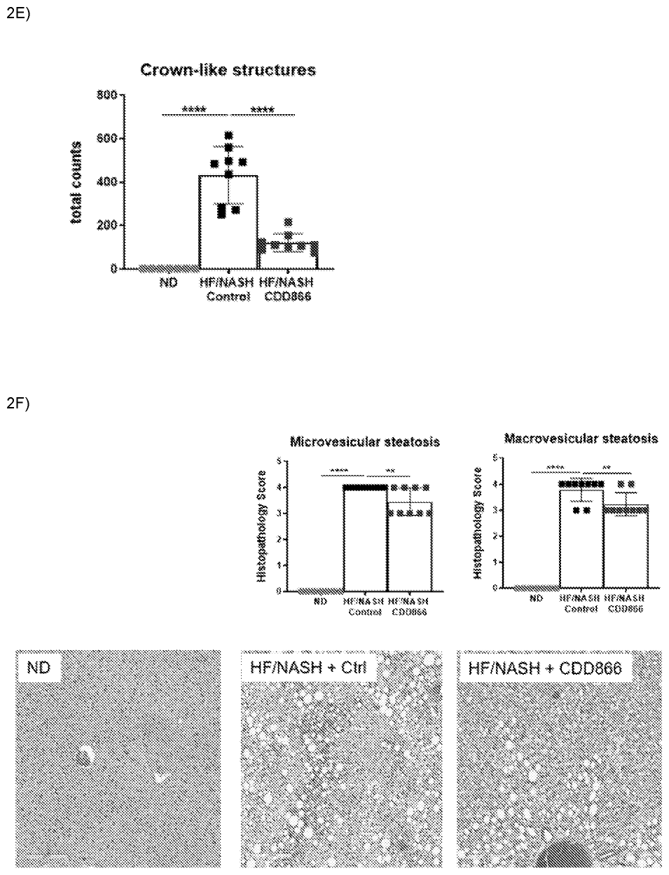

PicroSirius Red staining of liver sections at week 20 demonstrated that treatment with CDD866 significantly decreased liver fibrosis by 30% over control treatment (FIGS. 2A and 2B). Histopathological semi-quantitative scoring of PicroSirius-red stained liver sections did not reveal statistically significant changes although a trend towards reduction of fibrosis was observed in CDD866 treated livers (FIG. 2C).

CDD866 treatment also decreased myofibroblast marker a-SMA-positive staining in liver (−30% vs Ctrl) (FIG. 2D) and IBA1-positive hepatic crown like structures were significantly decreased in CDD866-treated livers (FIG. 2E).

Histopathological semi-quantitative scoring of hematoxylin eosin-stained liver sections revealed a significant decrease in liver micro- and macrovesicular steatosis upon CDD866 treatment (FIG. 2F).

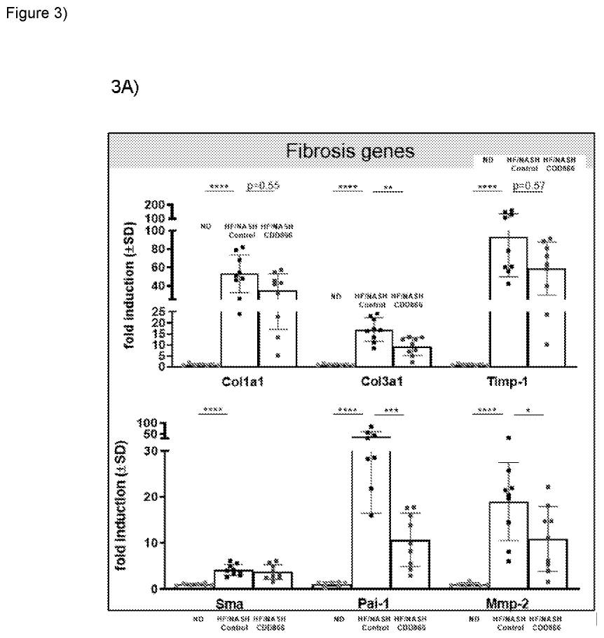

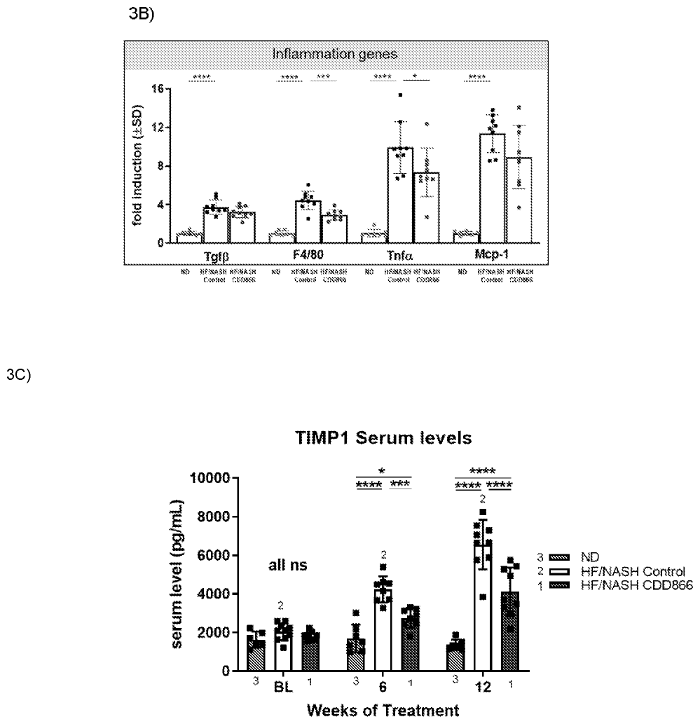

This observation was further confirmed by the decrease in the gene expression of liver fibrosis markers (FIG. 3A) and liver inflammatory markers F4/80 and TNFα (FIG. 3B). Also observed was a decrease in serum TIMP1 (FIG. 3C) and PIIINP (FIG. 3D) from 6 weeks of treatment (−36%/−19% vs Ctrl), which remained stable over time (−38%/−24%, end of study).

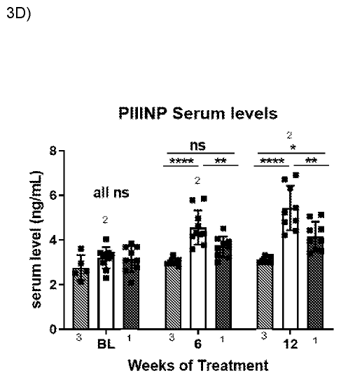

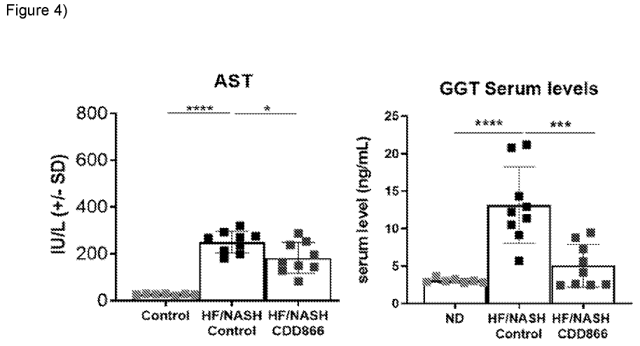

In blood analysis, a decrease of serum AST (−27%) and GGT (−62%) levels was observed at week 20 HF/NASH (FIG. 4).

### Example 2: DAX19 In Vivo 2—CCl₄-Induced Liver Fibrosis Model

Male C57BL/6J eight weeks of age were housed in a temperature- and humidity-controlled environment with 12-h light-12-h dark cycles and free access to standard rodent chow (Kliba-Nafag, Kaiseraugst, Switzerland) and tap water. To induce fibrosis, mice were be subjected to CCl4 application for 4 weeks, 3 times/week intraperitoneally (i.p.). Animals were randomized in three groups of mice: Group 1) Control: olive oil, i.p., 5 ml/kg, 3 times/week (n=12), group 2) CCl4, i.p., 5 ml/kg, 15% CCl4, 3 times/week+Control Antibody s.c., q7d (n=12) and group 3) CCl4, i.p., 5 ml/kg, 15% CCl4, 3 times/week+CDD866 30 mg/kg, s.c., q7d (n=10). Bodyweight was measured three times per week and fat and lean masses were measured at 0, 2 and 4 weeks using a mouse body composition nuclear magnetic resonance (NMR) analyzer (Minispec LF50; Bruker Optics, Germany).

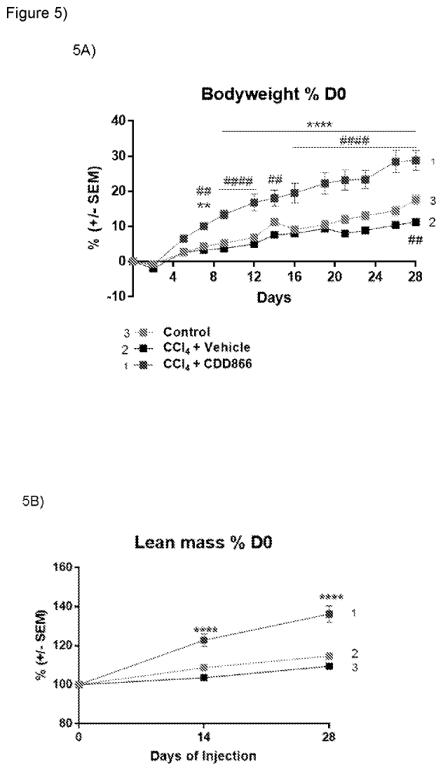

As shown in FIG. 5, an increase in body weight (FIG. 5A) and lean mass (FIG. 5B) was observed upon CDD866 treatment. Also observed was a decrease in total fat mass at day 14 and day 28 (FIG. 5C). This decrease in total fat mass was accompanied by epididymal white adipose tissue (WAT) decrease (FIG. 5D) and increase in brown adipose tissue (BAT) at day 28 (FIG. 5E).

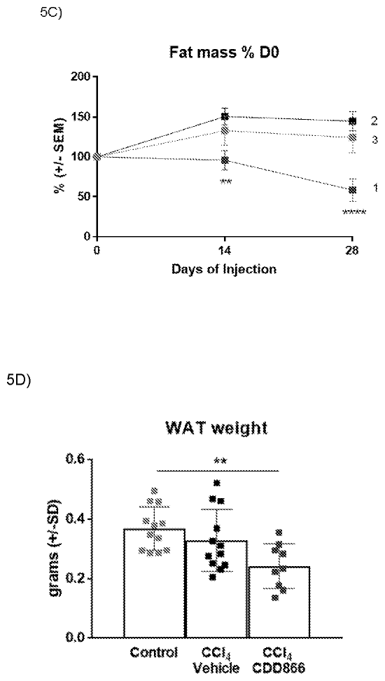

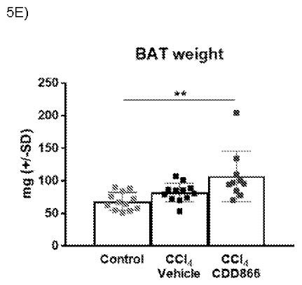

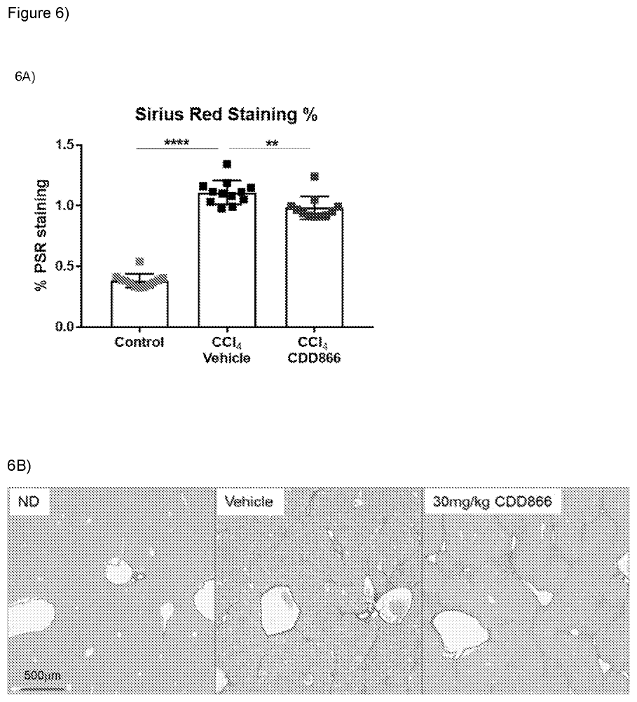

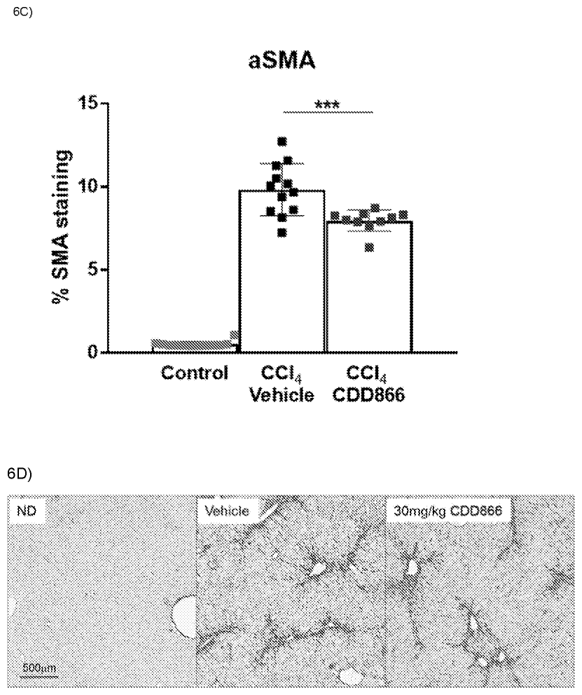

PicroSirius Red staining of liver sections at week 4 demonstrated that treatment with CDD866 significantly decreased liver fibrosis by 11% over control treatment (ND) (FIGS. 6A and 6B). Also observed was a significant decrease in liver anti-smooth muscle antibodies (aSMA) in mice treated with CDD866 (−16% vs Veh) (FIGS. 6C and 6D). A significant increase in serum fibrosis biomarkers, TIMP-1 and PIIINP, was observed in CDD866-treated animals (FIG. 6E).

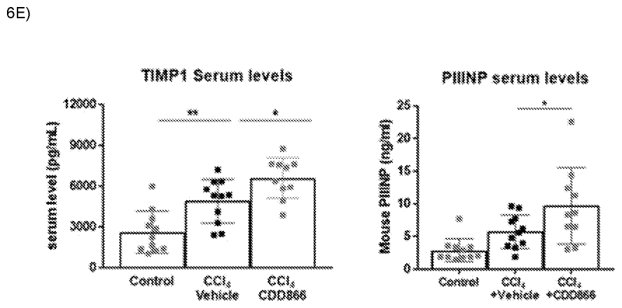

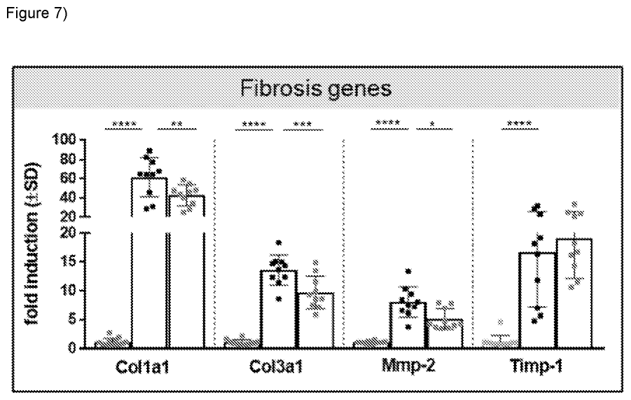

These observations were further confirmed by the levels of gene expression: at week 4 a decrease of around 30-40% was observed for Col1a1, Col3a1 and Mmp-2, while an increase was observed for Timp-1 (FIG. 7).

### Conclusion

Using the ActRII antagonist CDD866 (murinized BYM338) in two different models it can be shown that liver fibrosis is significantly reduced upon administration of the ActRII antagonist.

### Example 3: Clinical Study

### Study Design

BYM338X2211 was a non-confirmatory, randomized, subject and investigator blinded, placebo-controlled, parallel arm study, investigating a 48-week treatment period with intravenous bimagrumab in overweight/obese patients with type 2 diabetes. The study was registered with ClinicalTrials.gov Identifier NCT03005288. 75 patients were enrolled and randomized. For patients who provided consent for the optional MRI, their liver, visceral and subcutaneous fat content were assessed.

### Key Inclusion Criteria

Male and female, age 18 to 75 years (inclusive), in stable health condition as determined by past medical history, physical examination, vital signs, electrocardiogram, and laboratory tests at screening.
Type 2 diabetes (T2D), with an HbA1c between 6.5% and 10% (inclusive) at screening, on either metformin and/or DPP4 inhibitor agent, or no background therapy for T2D, with stable treatment for approximately 3 months prior to randomization.
Body mass index (BMI) of 28 to 40 kg/m² (inclusive) at screening.
Body weight between 65 and 140 kg (inclusive) at screening, and with a stable body weight (±5 kg) by history (patient report) and stable physical activity within 3 months prior to screening.
At screening vital signs should be as follows: oral body temperature between 35.0-37.5° C., systolic blood pressure 90 to 150 mm Hg, diastolic blood pressure 50 to 90 mm Hg, pulse rate, 50-100 bpm

### Key Exclusion Criteria

Pregnant or nursing (lactating) women, where pregnancy is defined as the state of a female after conception and until the termination of gestation confirmed by a positive hCG laboratory test.
Women of childbearing potential, defined as all women physiologically capable of becoming pregnant, unless they are using highly effective methods of contraception during dosing and for 6 months after stopping of investigational drug.
Diabetes other than Type 2 such as Type 1 diabetes, surgically induced-diabetes; “brittle” type 2 diabetes as per investigator judgment, history of severe hypoglycemic episodes in the year preceding screening, or hypoglycemic unawareness.
Abnormal liver function tests such as AST, ALT, alkaline phosphatase or serum bilirubin, or abnormal lipase and/or amylase.
History of clinically significant arrhythmias, unstable angina, myocardial infarction or stroke, coronary artery bypass graft surgery, or percutaneous coronary intervention (e.g. angioplasty or stent placement), within 6 months of screening or 1 year for drug-eluting stents.
Use of any anti-obesity medications, nutritional supplements or over the counter products for weight loss within 3 months of screening. Use of medications known to induce weight gain such as some anti convulsant and psychotropic medications (e.g. clozapine) within 3 months of screening.

### Screening (Days −21 to −8)

Participants underwent an onsite screening visit to determine their eligibility for the study. Subjects who qualified for enrollment following screening were scheduled for baseline assessments.

Lifestyle interventions included dietary counseling for weight loss with a daily caloric deficit of 500 kcal, with a diet that followed the American Diabetes Association (ADA) guidance for optimal glycemic control, and with protein intake of at least 1.2 g/kg/day to support muscle anabolism and to compensate for the caloric deficit. Patients received counseling for physical activity and were encouraged to follow established guidelines (American Diabetes Association, Starter Walking Plan, Adapted from I Hate to Exercise, 2^{nd} edition, by Charlotte Hayes). These interventions were initiated at screening once eligibility was confirmed. Trials with anti-obesity agents have to demonstrate treatment benefits on body weight/composition on a background of first line-therapy with lifestyle interventions. The daily caloric deficit of 500 Kcal is a standard approach and was expected to induce weight loss over the treatment period. The American Diabetes Association (ADA) walking program is tailored to the type of population in this study and is a gentle, easy to implement approach for physical activity. Exercise is known to enhance the effect of bimagrumab on muscle function, supporting the treatment benefit of bimagrumab on body composition and weight.

### Baseline (Days −7 to −1)

Prior to dosing (Day 1), patients who were eligible for enrollment following screening returned to the clinic to undergo baseline assessments.

Baseline scans were conducted prior to the Day 1 dose of the active drug or placebo, and included determination of hepatic fat content, abdominal subcutaneous fat and abdominal visceral fat by magnetic resonance imaging (MRI), and as well as determination of body composition by dual-energy X-ray absorptiometry (DXA). Additional baseline patient evaluation included anthropometric measurements (height, body weight, waist circumference, hip circumference, waist to hip ratio and body mass index (BMI) (Body weight (kg)/[Height (m)]²). Other baseline measurements included fasting glucose and insulin and determination of HbA1c.

### Randomization and Dosing (Day 1)

Eligible patients, based on screening and baseline assessments, were randomized in a 1:1 ratio to receive either bimagrumab or placebo. Randomization was stratified according to baseline BMI into 2 strata:

BMI between 28 kg/m² and 33 kg/m² (inclusive) and

BMI above 33 kg/m² and up to 40 kg/m² (inclusive)

Patients were assigned to one of the following 2 treatment arms in a ratio of 1:1

BYM338 (bimagrumab) 10 mg/kg up to maximum 1200 mg monthly (12 doses)

Placebo monthly (12 doses)

Administration of bimagrumab or placebo was done via an intravenous infusion over 30 minutes followed by an observation period that included safety and tolerability and PK sampling.

### Treatment Period (Days 1-336)

Patients continued on background standard of care to avoid a deterioration in glycemic control as per eligibility criteria (see key inclusion criteria above) throughout the study, enabling the evaluation of added treatment benefit with bimagrumab on glycemic parameters. T2D treatment is restricted to specific therapies for homogeneity of the study population and to enable interpretability of the data. Oral diabetes therapy with metformin and/or DPP4 inhibitor supported selection of patients who are early in their disease state and thus without significant co-morbidities. Moreover, these medications were less likely to affect body weight and thus confound the study results. If improvement in glycemic control was observed during the study, reduction in anti-diabetic treatment was allowed to prevent hypoglycemia.

Administration of bimagrumab or placebo was done via an intravenous infusion over 30 minutes followed by an observation period once every 4 weeks for a total of twelve doses. Bimagrumab was dosed based on body weight at 10 mg/kg, with a dose cap of 1200 mg for body weight equal to and above 120 kg. Placebo was provided as D5W, 5% Dextrose solution.

Patients received regular monitoring and advice on diet and physical activity as part of their monthly site visits throughout the study.

Patients were asked to return to the Investigator site for dosing approximately every 4 weeks during the treatment period. During these visits, patients were evaluated for safety, tolerability, PK and efficacy.

The treatment period ended 4 weeks after the last administration (on Day 308/Week 44).

### Follow Up (Days 364-392)

After completion of the treatment period patients had a follow-up period of 8 weeks with regular monitoring for safety and efficacy (at Week 52) until the end of study visit (EOS) which took place at 56 weeks, 12 weeks after the last study drug administration.

### Dose Rationale

In healthy volunteers (HV) and sIBM patients, a 10 mg/kg dose of bimagrumab was shown to provide exposure levels (i.e. above 10 μg/mL) at which the anabolic effect is observed and maintained over dosing intervals of 4 weeks [CBYM338X2102 (N=6 subjects), CBYM338X2104 (N=47 subjects)], for up to six doses [CBYM338X2109 (N=35 subjects)] and up to one year [CBYM338B2203 (N=54 sIBM patients)]. The threshold for minimal target exposure for bimagrumab is approximately 10 μg/mL, a concentration below which nonlinear clearance is observed, suggesting loss of full receptor saturation and target-mediated drug disposition. In clinical studies to date, bimagrumab concentrations approximately at or above 10 μg/mL for at least 4 weeks in HV and more than one year in sIBM patients has been safe, well tolerated, and demonstrated an increase in thigh muscle volume. The 26-week toxicology studies in cynomolgus monkeys showed a chronic exposure at NOAEL (300 mg/kg/week) of approximately 300-fold and 55-fold for AUC and Cmax respectively when compared to human exposures at 10 mg/kg at steady state.

Dosing in this study was weight-based for patients with body weight up to 120 kg, and was capped at 1200 mg for patients with body weight between 120 kg and 140 kg. Body-weight based dosing has proven to reduce variability in exposure in subjects/patients, and was implemented as applicable. Capped dose is selected for body weights >120 kg because of the uncertainty of the effect of large body weight and body composition (% fat mass vs. % lean mass) on the pharmacokinetics, exposure, and safety profile of bimagrumab. To date, the pharmacokinetic data is limited in obese subjects, and the maximal body weight in dosed subjects has been 116 kg, in studies conducted with bimagrumab in overweight to obese subjects with insulin resistance (N=10), and obese healthy subjects (N=6). The maximal amount of bimagrumab administered to-date is 3500 mg (at a dose of 30 mg/kg), given i.v. and as a single dose for a maximal body weight of 116 kg. This dose did not show over-exposure and caused no safety concerns. A capped dose for these subjects was selected to avoid over-exposure and to maintain bimagrumab levels around the threshold for safe anabolic effects over 4-week dosing intervals. Specifically, the selected amount of 1200 mg translates to a body weight based dose ranging from 10 to 8.6 mg/kg for the body weight range of 120-140 kg, which is predicted to result in exposure levels within the safe and efficacious range for bimagrumab and with minimal risk of over-exposure.

### Rationale for Duration of Treatment

A treatment duration of 48 weeks was selected to capture the temporal profile as well as maximal effect of bimagrumab on body fat mass. While a ceiling effect is typically observed on lean mass gain with bimagrumab, the loss of fat mass does not seem to plateau over a period of 24 weeks and even up to 64 weeks.

### Rationale for Follow-Up Period

The extended follow-up period of 8 weeks was selected to monitor the durability of treatment effect of bimagrumab on body fat mass, lean mass and glycemic control off treatment. The EOS visit being performed 12 weeks after the last administration covers the wash-out period of bimagrumab exposure associated with anabolic effect (approximately 8 weeks).

### Glucose Control Assessment

Fasting glucose and insulin were measured at different times.

HbA1c

HbA1c reflects average glucose concentrations over the past 3 months and therefore provided a useful index of the glycemic control of bimagrumab over that time period. It is a standard endpoint used to assess the glycemic efficacy of any anti-diabetic medication. HbA1c is a key glycemic parameter which correlates with reduction of risk of diabetic complications.

HOMA2-IR

Patients underwent fasting insulin and glucose assessment at screening to estimate the degree of insulin resistance using the homeostatic assessment model of insulin resistance (HOMA2-IR) and inverse of the HOMA2-IR.

QUICKI

QUICKI is being evaluated as it is a preferred estimate of insulin resistance than HOMA2-IR in patients with diabetes and elevated fasting glucose levels, e.g. >170 mg/dl (Yokoyama et al (2004) J. Clin. Endocrinol. Metab. p. 1481). QUICKI is a derived value of insulin sensitivity index using fasting glucose and insulin levels and provides additional and complementary information to that obtained with HOMA2-1R (Hrebicek et al (2002) J. Clin. Endocrinol. Metab. p. 144-7).

### Imaging

DXA Scan

Dual energy X-ray absorptiometry (DXA) is used to assess changes in body composition, including total fat and lean body mass (FBM and LBM) and appendicular skeletal fat and muscle mass (aFBM and aLBM). DXA instruments use an x-ray source that generates and is split into two energies to measure bone mineral mass and soft tissue from which fat and fat-free mass (or lean body mass) are estimated. The exam is quick (˜5-6 min), precise (0.5-1%) and non-invasive. DXA scanners have the precision required to detect changes in muscle mass as small as 5%.

MRI Scan

Magnetic resonance imaging (MRI) is used to assess changes in the percentage of fat in the liver (% fat fraction or % FF), the visceral and subcutaneous adipose tissue volumes in the abdominal region, as well as the paravertebral muscle cross-sectional area and associated fat contents (both the inter-muscle adipose tissue-IMAT and muscle FF contents). All images were acquired in the axial plane by using an imaging pulse sequences optimized for water/fat separation and adapted to the MRI system capabilities.

### Analysis of the Primary Variables

The primary aim of the study was to assess the effect of bimagrumab on total body fat mass. The primary efficacy variable was the change from baseline in fat mass at Week 48.

The study design enabled evaluation of efficacy based on the following dual criteria 1) statistical significance (superior treatment effect, 1-sided 10% level) in fat mass; and 2) clinical relevance of the change in fat mass (estimated median treatment effect of 5% or more). Weight loss of 5% has been shown to translate into clinical benefit in an overweight/obese population with T2D (Franz et al (2015) J. Acad. Nutr. Diet. p. 1447-1463). The randomization was stratified by BMI category (28 kg/m² and 33 kg/m², >33 kg/m² to 40 kg/m²) in order to achieve an approximate balance of BMI distribution across the two treatment groups. The cutoff value of 33 kg/m² represented the expected median BMI in that population.

A longitudinal mixed effects model was used with change from baseline in kg fat mass as the dependent variable, treatment arm, time, and a time*treatment interaction as fixed effects. Baseline fat mass and baseline BMI values were included in the model as covariates. Time was modeled as a categorical variable and unstructured within-subject covariance was used. Data collected from both randomization strata (BMI category at randomization) was included in the model. The change from baseline (absolute and %) in kg fat mass at Week 48 was estimated from this model. As a supportive analysis, the proportion of patients reaching at least 5% fat loss at Week 24 and Week 48 was presented by treatment group.

### Analysis of Secondary Variables

A secondary efficacy variable is the change in HbA1c at Week 24 and Week 48. Other parameters of glucose control and insulin sensitivity (fasting glucose and insulin, HOMA2-IR, QUICKI, Matsuda Index) and anthropometric body measurements (body weight, BMI, waist circumference, waist-to-hip ratio and lean body mass (LBM) as measured by DXA) are other secondary efficacy variables. Body fat mass as measured by DXA at week 24 was also a secondary efficacy variable.

The secondary variable of HbA1c was analyzed in a similar fashion to fat mass, to assess the statistical significance (superior treatment effect, 1-sided 10% level) of bimagrumab therapy on HbA1c, and the clinical relevance of this effect (median treatment effect of 0.5%). A model was used to describe HbA1c over time and the change in HbA1c at all time points of interest (including Week 48) was estimated from that model. The analysis considered observations censored after a change in background anti-diabetic medication or dose. This analysis was expected to be unbiased because adjustments for background medication/dose were based on observed data (HbA1c, FPG), making the censored data following the medication change likely missing at random (MAR). As a supporting analysis for metabolic changes, summaries of increase (and decrease) in background anti-diabetic medication may be done. A change in background anti-diabetic medication was defined as a change in daily dose and/or the addition of a second agent.

### Results

Trial Population. Enrolled subjects in this study were primarily Caucasian (76%) or black/African American (20%). All subjects were overweight or obese (mean±standard deviation BMI 32.9±3.4 kg/m², range: 28-40), with an approximately equal number of female (47%) and male (53%) subjects overall. All subjects had type 2 diabetes; mean HbA_{1c} was 7.99% (±1.025) and 7.66% (±0.950) in the bimagrumab and placebo group, respectively. A total of 78 subjects were enrolled, 37 in the bimagrumab group, of which 14 were male (38%) and 23 female (62%) and 38 in the placebo group, of which 26 were male (68%) and 12 female (32%). Key baseline laboratory values were comparable between treatment groups. Mean body weight was 6.76 kg lower in the bimagrumab group as compared to placebo. This difference is accounted for by the fact that the bimagrumab group had a greater percentage of female patients as compared to placebo.

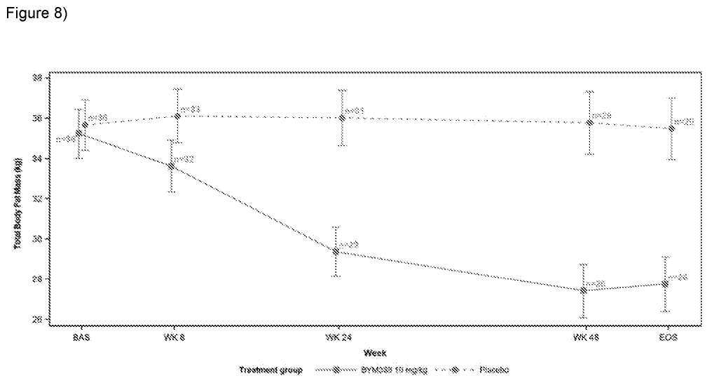

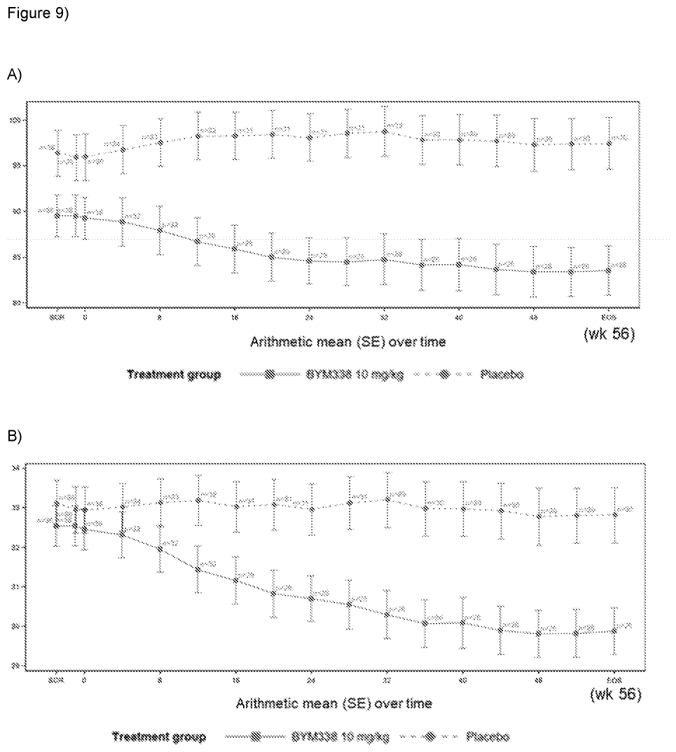

Body composition changes. A significant treatment effect of bimagrumab was observed on body composition at week 24 and week 48 as measured by dual energy X-ray absorptiometry (DXA) scan. A significant reduction in total fat mass was observed in the bimagrumab group when compared to placebo at both the wk 24 and wk 48 time point: −15.0% (−5.2 kg) in subjects on bimagrumab vs. placebo at wk 24, and −20.5% (7.3 kg) in subjects on bimagrumab vs. placebo at wk 48 (all p<0.001, FIG. 8). This effect was observed as early as Week 8 (first post-dose scan) and persisted until the end of the study at week 56. A significant reduction in body weight was also observed at Week 48 in the bimagrumab group when compared to placebo: −6.5% (5.9 kg) in BYM338 vs. −0.8% (0.8 kg) in placebo, p<0.001 (FIG. 9A), translating into a decrease in BMI of −6.7% (2.2 kg/m²) in BYM338 vs. −0.8% (0.3 kg/m²) in placebo, p<0.001 (FIG. 9B). These effects persisted until the end of the study at week 56.

Insulin sensitivity and HbA_{1c}. The treatment effect of bimagrumab on HbA1c showed a reduction of 0.76% [80% Cl −1.05; 0.48] vs an increase of 0.04% [80% Cl −0.23; 0.31] in placebo at Week 48 (p=0.005). Insulin sensitivity was measured based on both fasting insulin and glucose and on a meal tolerance test. The treatment effect of bimagrumab on insulin sensitivity showed a significant improvement as measured by QUICKI (bimagrumab+0.01; placebo, no change, p=0.033) at week 36. There was a trend toward improvement in insulin sensitivity as measured by both the Matsuda index (bimagrumab +3.15, placebo +1.78, p=0.099) at week 48 and HOMA2-IR (bimagrumab −0.09; placebo +0.57, p=0.081).

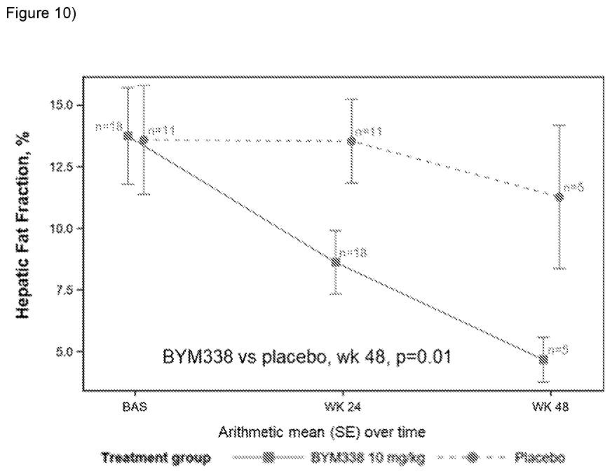

Fat distribution. A significant treatment effect with bimagrumab was observed on fat distribution at week 24 and week 48 (FIG. 10). A significant reduction in hepatic fat fraction (HFF) was observed at week 24 in the bimagrumab group when compared to the placebo group with −4.6 percentage point over 24 weeks in the bimagrumab group vs. +0.23 percentage points in the placebo group, p=0.006. At week 48 a significant reduction in HFF was observed in the bimagrumab group when compared to the placebo group with 51.9% (−7 percentage points) over 48 weeks in in the bimagrumab group vs. 18.3% (2.3 percentage points) in the placebo group, p=0.01. The number of subjects who had an MRI at week 48 is smaller than at the prior time points because the original protocol had not included an MRI at week 48. An MRI at this time point was added after the interim analysis was completed, and after some subjects had already completed the study and were no longer eligible to receive this assessment.

A significant reduction in abdominal visceral fat was observed at week 24 in the bimagrumab group when compared to placebo with a reduction of 1.49 L in the bimagrumab group versus 0.22 L in the placebo group, p>0.001 and at 48 weeks of −34.5% (1.5 L) in BYM338 vs. −0.2% (0.01 L) in placebo, p=0.08.

### Discussion

In the current study, weight loss of just 7% was accompanied by a reduction in hepatic fat of 52% in subjects on bimagrumab vs. 18% in subjects on placebo (both groups included a diet and exercise intervention). This amount of hepatic fat loss is an unexpected finding based on prior observations in patients having undergone bariatric surgery (Phillips et al (2007) Diabetes, Obesity and Metabolism, 10, 2008, 661-667) or dietary intervention (Lewis et al (2006) Obesity Surgery, 16, 697-701), in which 10% weight loss, on average, results in approximately 30% loss of hepatic fat. In addition to a reduction in hepatic fat, treatment with bimagrumab also reduced total body fat mass (primarily visceral fat), waist circumference and HbA1c, all while increasing lean body mass. These findings are important as both type 2 diabetes and insulin resistance are strong predictors for progression of NAFLD/NASH to fibrosis and cirrhosis; reversing fatty liver will reduce the risk of incident type 2 diabetes and metabolic syndrome (reviewed in Cernea et al Expert Rev Clin Pharmacol 2017). Furthermore, increased lean mass that results from bimagrumab therapy is important because patients with NASH are at increased risk of developing sarcopenia, which is the age-related loss of muscle mass and function (Koo et al J Hepatology 2017; Petta et al. Ali Pharma Thera 2017; Carias et al. J Gastroenterol Hepatol 2016). Bimagrumab addresses many of the metabolic abnormalities commonly found in people with obesity, T2D and NASH.

What is claimed is:

1. A method for the treatment or prevention of liver disease or disorder in a subject in need thereof, comprising administering to said subject a therapeutically effective amount of an activin receptor type II (ActRII) antagonist.

2. A method for slowing, arresting, or reducing the development of a chronic liver disease or disorder, e.g. NAFLD, non-alcoholic steatohepatitis (NASH), or liver fibrosis, in a subject in need thereof, comprising administering to said subject a therapeutically effective amount of an activin receptor type II (ActRII).

3. The method according to any one of the preceding claims, wherein said subject has at least one condition selected from hepatic steatosis, lobular inflammation, and hepatocellular ballooning.

4. The method according to any one of the preceding claims, wherein said subject has hepatic steatosis.

5. The method according to any one of the preceding claims, wherein said liver disease or disorder is non-alcoholic fatty liver disease (NAFLD).

6. The method according to any one of the preceding claims, wherein said liver disease or disorder is non-alcoholic steatohepatitis (NASH).

7. The method according to any one of the preceding claims, wherein said liver disease or disorder is liver fibrosis.

8. The method according to any one of the preceding claims, wherein administration of a therapeutically effective amount of the ActRIIA/ActRIIB antagonist to said subject reduces the hepatic fat fraction in said subject compared to the hepatic fat fraction in said subject prior to the administration of a therapeutically effective amount of the ActRIIA/ActRIIB antagonist.

9. The method according to any one of the preceding claims, wherein administration of a therapeutically effective amount of said ActRIIA/ActRIIB antagonist reduces NAFLD Activity Score (NAS) by at least 1 point, at least 2 points or at least 3 points.

10. The method according to any one of the preceding claims, wherein administration of a therapeutically effective amount of said ActRIIA/ActRIIB antagonist reduces at least one of hepatosteatosis, hepatic inflammation and hepatocellular ballooning by at least 1 NAS point.

11. The method according to any one of the preceding claims, wherein said subject is a diabetic subject, an obese subject, or a subject with metabolic syndrome or another metabolic disorder.

12. The method according to any one of the preceding claims, wherein said subject has type 2 diabetes.

13. The method according to any one of the preceding claims, wherein said subject is concomitantly receiving standard of care treatment for Type 2 diabetes.

14. The method according to claim 13, wherein the standard of care treatment is selected from metformin, DPP4 inhibitor, metformin/DPP4 inhibitor, sulfonylureas, thiazolidinediones, GLP-1 receptor agonists, SGLT2 inhibitors, insulin therapy.

15. The method according to any one of the preceding claims, wherein the activin receptor type II (ActRII) antagonist is an ActRIIA and/or ActRIIB antagonist.

16. The method according to any one of the preceding claims, wherein the activin receptor type II (ActRII) antagonist is an ActRIIA and ActRIIB antagonist.

17. The method according to any one of the preceding claims, wherein the ActRIIA/ActRIIB antagonist is an anti-ActRII antibody or functional fragment thereof.

18. The method according to claim 17, wherein said ActRIIA/ActRIIB-binding antibody is selected from the group comprising:
a) an antibody comprising the three CDRs of SEQ ID NO:1, SEQ ID NO:2, SEQ ID NO:3;
b) an antibody comprising the three CDRs of SEQ ID NO:4, SEQ ID NO:5, SEQ ID NO:6;
c) an antibody comprising the three CDRs of SEQ ID NO:1, SEQ ID NO:2, SEQ ID NO:3 and the three CDRs of SEQ ID NO:4, SEQ ID NO:5, SEQ ID NO:6;
d) an ActRIIA/ActRIIB-binding antibody comprising a HC domain comprising SEQ ID NO:8;
e) an ActRIIA/ActRIIB-binding antibody comprising a LC domain comprising SEQ ID NO:7;
f) an ActRIIA/ActRIIB-binding antibody comprising a HC domain comprising SEQ ID NO:8 and a LC domain comprising SEQ ID NO:7.
g) an ActRIIA/ActRIIB-binding antibody comprising a VL domain comprising SEQ ID NO:9,
h) an ActRIIA/ActRIIB-binding antibody comprising a VH domain comprising SEQ ID NO:10,
i) an ActRIIA/ActRIIB-binding antibody comprising a VL domain comprising SEQ ID NO:9 and a VH domain comprising SEQ ID NO:10,
j) an antibody capable of binding to each of the following epitopes of ActRIIB:
(i) WLDDFN (SEQ ID NO:11) and
(ii) CEGEQDKRLHCYASW (SEQ ID NO:15).
k) an antibody capable of binding to each of the following epitopes of ActRIIB:
(i) WLDDFN (SEQ ID NO:11)
(ii) CEGEQDKRLHCYASW (SEQ ID NO:15) and
(iii) GCWLDDFNC (SEQ ID NO:12).
l) an antibody, which is:
(i) capable of binding to an epitope consisting of WLDDFN (SEQ ID NO:11) and
(ii) capable of binding to an epitope consisting of CEGEQDKRLHCYASW (SEQ ID NO:15).

19. The method according to any one of the preceding claims, wherein the ActRIIA/ActRIIB antagonist is bimagrumab.

20. The method according to claim 19, comprising administering about 3 mg/kg to about 10 mg/kg bimagrumab to said subject.

21. The method according to claim 19 or 20, comprising administering about 10 mg/kg bimagrumab to said subject.

22. The method according to any one of claims 19 to 21, wherein bimagrumab is administered every 4 weeks.

23. The method according to any one of claims 19 to 22, wherein bimagrumab is administered every 4 weeks for at least 3 months, at least 6 months, at least 9 months or at least 12 months.

24. The method according to any one of the preceding claims, comprising administering at least one further therapeutic agent.

25. The method according to claim 24, comprising administering the ActRIIA/ActRIIB antagonist in combination with at least one further therapeutic agent for the treatment or prevention of liver disease.

26. The method according to claim 25, wherein the at least one further therapeutic agent is FXR agonist (e.g., tropifexor, nidufexor, obeticholic acid (6α-ethyl-chenodeoxycholic acid), cilofexor (GS-9674, Px-102), TERN-101 (LY2562175), EYP001 (PXL007), EDP-305, AKN-083 (Allergan), INT-787 (Intercept), INT-767 (Intercept), AGN-242256 (Allergan), MET409 (Metacrine), Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, IVA337 (Inventive), CER-002 (Cerenis), GLP-1 agonist (e.g., exenatide, liraglutide, semaglutide, NC-101 (Naia Metabolic), G-49 (Astrazeneca), ZP2929 (BI/Zealand), PB-718 (Peg Bio), FGF agonist (e.g., pegbelfermin (ARX618), BMS-986171, NGM-282, NGM-313, YH25724, tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), FXR agonist (M480 (Metacrine), NTX-023-1 (Ardelyx), INV-33 (Innovimmune)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption) or any combination thereof.
